(19) United States
(12) Patent Application Publication
(10) Pub. No.: US 2023/0295395 A1
(43) Pub. Date: Sep. 21, 2023

(54) METHODS AND COMPOSITIONS FOR DEGRADING POLYMERS

(71) Applicant: Regents of the University of Minnesota, Minneapolis, MN (US)

(72) Inventors: Steven John Severtson, Minneapolis, MN (US); Drew Allen Hauge, Minneapolis, MN (US); Jiwei Zhang, Minneapolis, MN (US); Jesus David Castaño Urueña, Minneapolis, MN (US)

(21) Appl. No.: 18/122,081

(22) Filed: Mar. 15, 2023

Related U.S. Application Data
(60) Provisional application No. 63/319,908, filed on Mar. 15, 2022.

(51) Int. Cl. C08J 11/10; C12P 7/62; B09B 3/60

(52) U.S. Cl. CPC C08J 11/105; C12P 7/62; B09B 3/60; C08J 2351/08; B09B 2101/75

(57) ABSTRACT

Described herein are methods and compositions for degrading a polymer. For example, methods can include comprising contacting a polymer with one or more polymer-degrading fungal species, wherein the polymer comprises a polyester graft, and wherein the polymer is a polymerization product of at least a monomer selected from the group consisting of acrylates, methacrylates, and combinations thereof.

### CROSS-REFERENCE TO RELATED APPLICATIONS

This application claims the benefit of priority to U.S. Provisional Application Ser. No. 63/319,908, filed on Mar. 15, 2022. The disclosure of the prior application is considered part of the present application and is incorporated by reference in its entirety into the disclosure of the present application.

### TECHNICAL BACKGROUND

This disclosure relates to methods and compositions for degrading a polymer (e.g., a pressure sensitive adhesive (PSA) composition). Degradation of the polymer that can use one or more polymer-degrading fungal species.

### BACKGROUND

Disposable consumer products often contain polymers (e.g., pressure-sensitive adhesives (PSAs)) that are produced and formulated with fossil-derived chemicals, such as petroleum-derived monomers. Such materials can contribute to environmental pollution due to high recalcitrance (i.e. difficult degrading) and prevention of recycling other recyclable plastic and paper products when polymers and recyclable materials are co-mingled. There is a need for improved methods for degrading polymers, such as PSAs.

### SUMMARY

Disclosed herein are methods and compositions for degrading polymers, such as pressure-sensitive adhesive (PSA) composition.

Described herein are methods of degrading a polymer comprising contacting a polymer with one or more polymer-degrading fungal species, wherein the polymer comprises a polyester graft, and wherein the polymer is a polymerization product of at least a monomer selected from the group consisting of acrylates, methacrylates, and combinations thereof.

Also described herein are methods comprising contacting a polymer and one or more polymer-degrading fungal species, wherein the polymer comprises at least 15 wt. % of a polyester graft, wherein the polymer is a polymerization product of at least a monomer selected from the group consisting of acrylates, methacrylates, and combinations thereof, and wherein the polymer comprises a copolymerizable lactide-based macromonomer; and degrading the polymer with a predetermined time period.

Also described herein are methods of degrading a polymer comprising contacting a polymer with one or more polymer-degrading fungal species, wherein the polymer comprises a polyester graft, and wherein the polymer is a polymerization product of at least a monomer selected from the group consisting of acrylates, methacrylates, and combinations thereof; a hydrolytic polypeptide selected from the group consisting of a protease and a lipase; and an oxidative polypeptide selected from the group consisting of a laccase, a peroxidase, and a hydroxylase.

In some cases, in any of the methods or compositions described herein, the one or more polymer-degrading fungal species is selected from the group consisting of Pestalotiopsis chamaeropsis, Ganoderma lucidum, Gloeophylum trabeum, Lenzites betulina, Trametes pubescens, Trametes versicolor, Leucogyrophana olivascens, Plicaturopsis crispa, Fistulina hepatica, Armillaria sp., Lentinus edodes, Humicola grisea, Aurebasidium pullulans, Aspergillus niger, Penicillum sp., Coniophora puteana, Resinicium bicolor, Panellus stipticus, Trametes hirsuta, Ganoderma austrade, Fomitiporia mediterranea, and Pestalotiopsis micropsora.

In some cases, in any of the methods or compositions described herein, the one or more fungal species includes at least two fungal species selected from the group consisting of L. betulina and T. versicolor, L. betulina and T. pubescens, and P. microspora and T. versicolor.

In some cases, in any of the methods or compositions described herein, the polymer comprises at least 15 wt. % of the polyester graft. In some cases, in any of the methods described herein, the polyester graft is a macromonomer. In some cases, in any of the methods described herein, the macromonomer is a reaction product of at least a lactide monomer, a caprolactone monomer, or combinations thereof.

In some cases, in any of the methods or compositions described herein, the polymer is a pressure-sensitive adhesive. In some cases, in any of the methods or compositions described herein, the polymer is a latex product.

In some cases, in any of the methods described herein, the method comprises, following the contacting, degrading at least 40 wt. % of the polymer. In some cases, in any of the methods described herein, the degrading occurs in a controlled environment, wherein the controlled environment does not contain other carbon-containing materials. In some cases, in any of the methods described herein, the contacting further comprises adding wheat bran to the polymer and the polymer-degrading fungal species.

In some cases, in any of the methods described herein, the predetermined time period is from about 1 day to about 30 days.

In some cases, in any of the methods or compositions described herein, the hydrolytic polypeptide, the oxidative polypeptide, or both is derived from a polymer-degrading fungal species.

As used herein, “a” can include one or more of the indicated items. For example, “a polyester graft” can include one or more polyester grafts.

As used herein, the term “about” regard to an amount can include values slightly outside the cited values, e.g., plus or minus 0.1% to 10% of the indicated value. For example, about 15 wt. % includes 5 wt. % to 25 wt. %.

The details of one or more embodiments of the invention are set forth in the accompanying drawings and the description below. Other features, objects, and advantages of the invention will be apparent from the description and drawings, and from the claims. The features described below in separate embodiments can be combined in alternate embodiments.

### DESCRIPTION OF DRAWINGS

FIG. 1A is an exemplary schematic of the polymerization used generate a polyester graft (e.g., macromonomer (MM)), and an exemplary structure of a PSA. FIG. 1B is an exemplary schematic of a protocol used to test for PSA-degrading fungal strains.

FIG. 2 is an exemplary schematic of different physical, chemical, and biological tests that could be used to assess PSA-degrading ability of fungal strains.

FIG. 3 depicts exemplary results from a drop assay used to detect fungal candidates that were able to degrade PSAs.

FIG. 4 is a graph showing PSA degradation in a PSA-latex containing medium by a variety of fungal species.

FIG. 5 is a graph of CO₂ production from a variety of fungal species (left) and an exemplary depiction of the CO₂ measurement set-up (right).

FIG. 6 is a scanning electron microscope image of PSA-containing films degraded by fungi after 90 days of incubation.

FIGS. 7A-7B show a PSA formula and schematic design to test its biodegradability. FIG. 7A is an exemplary PSA polymer synthesis scheme. The PSA materials used for biodegradation studies were in latex and film form, as shown on the far right. FIG. 7B depicts an overview of an exemplary PSA biodegradation experimental set-up.

FIGS. 8A-8D depict screening fungal capacities to degrade PSAs. FIG. 8A depicts an exemplary PSA-agar plate screening test showing different fungi's ability to degrade the latex substrate by forming clear zones around fungal colonies. H. grisea is shown here as a representative. As listed in Table 2, 53 different fungi were screened using a drop assay for the ability to degrade PSA. FIG. 8B shows PSA removal (96%) by T. versicolor after 60 days. FIG. 8C is a graph showing the results of a liquid degradation assay (i.e., clearance of the PSA as measured via turbidity reduction of the liquid culture) over a 60-day incubation period for the 23 fungi selected by the plate screening step. Eight out of 23 fungi showed high PSA removal efficiency represented by a clearance of around 50% or higher in 60 days, namely T. versicolor (96%), P. microspora (96%), P. stipticus (88%), G. lucidum (77%), P. chamaeropsis (71%), L. betulina (64%), T. pubescens (60%), and H. grisea (48%). High degraders are shown in dark grey, while the other fungi evaluated are shown in light gray. FIG. 8D is a graph of the effects of MM concentration from 0 wt % to 70 wt % on biodegradation rates for T. versicolor and P. microspora. Plotted are mean values and standard deviations from three independent replicates.

FIGS. 9A-9C depict results from respirometry tests show fungi degraded and converted PSA films as a sole carbon source. FIG. 9A is an exemplary schematic of an experimental set-up for evaluating PSA-film fungal metabolism as a sole carbon source. FIG. 9B is a graph of cumulative CO₂ generated by fungal metabolism of PSA films over a 90-day cultivation period. FIG. 9C is a graph of PSA film weight loss after 90 days of fungal degradation and metabolism. Mean ± SD values are the product of three independent replicates; different letters indicate a significant difference (p<0.05) between fungal treatments.

FIGS. 10A-10B depict PSA film degradation characterization by SEM and FTIR. FIG. 10A are exemplary SEM images of PSA films after 90 days of incubation with eight fungi. Abundant fungal growth and signs of disintegration were apparent compared with the uninoculated sample (blank). Scale bar =40 μm. FIG. 10B is an FTIR-spectra of degraded and undegraded hybrid PSA with several bands labeled. The inset chart shows the transmittance of peaks associated with hydroxyl and carbonyl groups of a blank and degraded samples. The structure of the monomer unit of the undegraded hybrid PSA polymer is shown at the bottom right.

FIGS. 11A-11C depict the influences of fungal degradative enzymes on PSA degradation. FIG. 11A is a heatmap showing the enzyme activities of laccase, protease, esterase and lipase in the 23 fungal species tested. This figure used cumulative enzyme activity and PSA removal values over a 60-day cultivation period. (FIG. 12 shows additional enzyme activity changes over time.) Hierarchical clustering using the online tool heatmapper (heatmapper.ca/) used average linkage and Euclidean distance. FIG. 11B is a correlation matrix of PSA-degrading factors made with spearman correlation. Significant correlations are indicated by a star (*) (p-value<0.05). FIG. 11C depicts a multiple linear regression analysis, including the effect of protease and laccase on PSA removal. Both coefficients were statistically significant (p<0.05). This model accounted for approximately 35% of the variation in the biodegradation rate.

FIGS. 12A-12B depict enzyme activities as a function of time using liquid media assays. Protease, esterase, laccase, and lipase activities were assessed.

FIG. 13 depicts turbidity changes over time for the PSA samples inoculated with L. betulina, T. versicolor, T. pubescens, P. microspora and the indicated consortia. The values reported are the average of three replicates and the error bars correspond to the standard deviation.

FIG. 14 depicts a comparison of PSA clearance at day 15 during the PSA degradation experiment. The values reported are the average of three replicates and the error bars correspond to the standard deviation. Different letters indicate significant differences (p<0.05).

FIG. 15 is a graph of the PSA film weight loss after 100 days when degraded by various fungi and fungal consortia cultured on additional additives (e.g., different substrates such as wheat bran, wheat straw, etc).

FIG. 16 depicts a series of representative scanning electron microscope (SEM) images of PSA films subjected to the four different substrates of FIG. 15 with and without the consortium of P. microspora-T. versicolor present.

### DETAILED DESCRIPTION

Described herein are methods and compositions related to degrading polymers such as polymers including a polyester graft. For example, compositions used in the methods described herein can include cells or spores of one or more polymer-degrading fungal species.

Polymers are a material that can contain large molecules called macromolecules, composed of subunits, called monomers. Polymers, both natural and synthetic, are created via polymerization monomers. A polymer's large molecular mass, relative to the smaller molecular mass of the monomers, can produce unique physical properties including toughness, high elasticity, viscoelasticity, and amorphous and semicrystalline structures. Polymers range from synthetic plastics such as polystyrene to natural biopolymers such as DNA. In some cases, polymers described herein are liquid polymers. In some cases, the polymers described herein are solid (e.g., polymer films).

One example of a polymer is a pressure-sensitive adhesives (PSAs), which can be applied as a high-solids, low-viscosity, aqueous colloids (latex) that are water-based. In addition to being more convenient to handle and process than other PSA products, water-based PSAs are safer and more sustainable. PSAs, such as water-based PSAs can be found in the tape, label, and specialty packaging products. Such PSAs include a small percentage of formulation and processing chemicals (e.g., emulsifiers, viscosity modifiers, defoamers, etc.), but the majority of the content is polymers generated primarily from acrylic monomers. These adhesive polymers can be petroleum-based. Some of the polymer can be difficult to degrade (i.e., recalcitrant) because of their carbon-carbon backbones. Though PSAs constitute a relatively small fraction of disposable commercial products, their presence can impede the recycling and remediation of significant waste stream components. (See, for example, Droesbeke et al. Prog. Polym. Sci. 117: 101396).

Surprisingly, it was found that some fungal species can degrade polymers, in particular, grafted polymers, such as polymers comprising a polyester graft, such as a PSA containing lactide-based macromonomers (MMs). Also, several fungal species capable of secreting hydrolytic and oxidative enzymes degraded the polymers.

Compositions

Described herein are compositions that include one or more polymer-degrading fungal species. As described herein, the polymer-degrading fungal species may degrade the polymer in a degradation assay.

Also described herein are compositions that include at least one hydrolytic polypeptide selected from the group consisting of a protease and a lipase, at least one oxidative polypeptide selected from the group consisting of laccase, peroxidase, and hydroxylase, and a polyester graft.

In some cases, a composition includes cells or spores of a polymer-degrading fungal species described herein and any of the polymers described herein that includes at least 15 wt. % of a polyester graft described herein. In some cases, the composition includes at least 5 mg of the cells or spores of the polymer-degrading fungal species per 10000 mg of the polymer. For example, the composition includes at least about 10-100 mg of the cells or spores of the polymer-degrading fungal species per 400-10000 mg of the polymer.

In some cases, a composition includes at least one hydrolytic polypeptide described herein, at least one oxidative polypeptide described herein, and any of the polymers described herein.

Polymer-degrading fungal species. Fungi have been applied for bioremediation (i.e., mycoremediation) of diverse xenobiotics, including textile dyes, polycyclic aromatic hydrocarbons, and agricultural wastes. (See, for example, Jutinico-Shubach et al. Bioremediat. J. 26: 179-197). Fungi can also degrade synthetic plastic polymers such as polyurethane, polyethylene, polyvinyl chloride, and polystyrene; although, the effectiveness of the fungal degradation depends on the fungal species used and any accompanying physicochemical treatment methods. (See, for example, Srikanth et al. Bioresour. Bio process. 9: 42).

Described herein are one or more fungal species that degrade any of the polymers described herein. In some cases, a polymer-degrading fungal species that degrades the polymer described herein is identified by a degradation assay (e.g., a drop assay, a liquid degradation assay, a weight loss assay, or a growth-on-polymer assay). In some cases, the polymer-degrading fungal species can be preserved (e.g., lyophilized). In some cases, the polymer-degrading fungal species can be provided as fungal spores.

Non-limiting examples of polymer-degrading fungal species include polymer-degrading fungal species of a genera selected from the group consisting of Armillaria, Aspergillus, Aurebasidium, Coniophora, Fistulina, Fomitiporia, Ganoderma, Gloeophylum, Humicola, Lentinus, Lenzites, Leucogyrophana, Panellus, Penicillum, Pestalotiopsis, Plicaturopsis, Resinicium, and Trametes.

Non-limiting examples of polymer-degrading fungal species can include polymer-degrading fungal species selected from the group consisting of Pestalotiopsis chamaeropsis, Ganoderma lucidum, Gloeophylum trabeum, Lenzites betulina, Trametes pubescens, Trametes versicolor, Leucogyrophana olivascens, Plicaturopsis crispa, Fistulina hepatica, Armillaria sp., Lentinus edodes, Humicola grisea, Aurebasidium pullulans, Aspergillus niger, Penicillum sp., Coniophora puteana, Resinicium bicolor, Panellus stipticus, Trametes hirsuta, Ganoderma austrade, Fomitiporia mediterranea, and Pestalotiopsis micro psora.

In some cases, the one or more polymer-degrading fungal species or cells or spores of a polymer-degrading fungal species is selected from the group consisting of Pestalotiopsis chamaeropsis, Ganoderma lucidum, Gloeophylum trabeum, Lenzites betulina, Trametes pubescens, Trametes versicolor, Leucogyrophana olivascens, Plicaturopsis crispa, Fistulina hepatica, Armillaria sp., Lentinus edodes, Humicola grisea, Aurebasidium pullulans, Aspergillus niger, Penicillum sp., Coniophora puteana, Resinicium bicolor, Panellus stipticus, Trametes hirsuta, Ganoderma austrade, Fomitiporia mediterranea, and Pestalotiopsis micropsora.

In some cases, the polymer-degrading fungal species or cells or spores of a polymer-degrading fungal species is selected from the group consisting of Pestalotiopsis chamaeropsis, Ganoderma lucidum “stramets”, Gloeophylum trabeum ATCC 11539, Lenzites betulina ATF03, Trametes pubescens Ban020, Trametes versicolor A1 0ATF, Leucogyrophana olivascens ATCC 22108, Plicaturopsis crispa FD-325.553, Fistulina hepatica ATCC 644Error! Reference source not found.28, Armillaria sp., Lentinus edodes ATCC 48883, Humicola grisea ATCC 16298, Aurebasidium pullulans ATCC 9348, Aspergillus niger ATCC 6275, Penicillum sp. Wall BD, Coniophora puteana ATCC 12675, Resinicium bicolor ATCC 64897, Panellus stipticus, Trametes hirsuta ATCC 34679, Ganoderma austrade ATCC 90302, Fomitiporia mediterranea MF, Pestalotiopsis micropsora, and combinations thereof. In some cases, the polymer-degrading fungal species is a fungal species described in Table 2.

In some cases, compositions can include one or more polymer-degrading fungal species, or cells or spores of a polymer-degrading fungal species selected from the group consisting of Pestalotiopsis chamaeropsis, Ganoderma lucidum “stramets”, Gloeophylum trabeum ATCC 11539, Lenzites betulina ATF03, Trametes pubescens Ban020, Trametes versicolor A1 ATF, Leucogyrophana olivascens ATCC 22108, Plicaturopsis crispa FD-325.553, Fistulina hepatica ATCC 644Error! Reference source not found.28, Armillaria sp., Lentinus edodes ATCC 48883, Humicola grisea ATCC 16298, Aurebasidium pullulans ATCC 9348, Aspergillus niger ATCC 6275, Penicillum sp. Wall BD, Coniophora puteana ATCC 12675, Resinicium bicolor ATCC 64897, Panellus stipticus, Trametes hirsuta ATCC 34679, Ganoderma austrade ATCC 90302, Fomitiporia mediterranea MF, and Pestalotiopsis micropsora; and any of the polymers described herein.

In some cases, the one or more polymer-degrading fungal species are combinations of polymer-degrading fungal species (e.g., two, three, four, five, six, seven, eight, nine, ten, eleven, twelve, thirteen, fourteen, fifteen, sixteen, seventeen, eighteen, nineteen, twenty, twenty-one, or twenty-two polymer-degrading fungal species) can degrade any of the polymers described herein. In some cases, the one or more polymer-degrading fungal species are combinations of two polymer-degrading fungal species. The combination of two polymer-degrading fungal species can include L. betulina ATF03 and T. versicolor A1 ATF, L. betulina ATF03 and T. pubescens Ban020, or P. microspora and T. versicolor A1 ATF. In some cases, the one or more polymer-degrading fungal species are combinations of two polymer-degrading fungal species selected from the group consisting of L. betulina ATF03 and T. versicolor A1 ATF, L. betulina ATF03 and T. pubescens Ban020, and P. microspora and T. versicolor A1 ATF.

Fungal species can be identified in a variety of ways, including sequencing the ITS region. The ITS region (Internal Transcribed Spacer region) is a nucleic acid sequence located in the rRNA gene transcription region that corresponds to the large and small subunit rRNA of the polycistronic rRNA precursor. The ITS sequence used for fungal identification usually includes ITS1, 5.8 S and ITS2. The length of the fungal ITS region is generally 500-750 base pairs. The ITS region, or parts thereof, can be sequenced using any standard sequencing technology and then aligned to determine a percent sequence identity.

The terms “sequence identity” or “identity” as used herein with respect to polynucleotide or polypeptide sequences refer to the nucleic acid residues or amino acid residues in two sequences that are the same when aligned for maximum correspondence over a specified comparison window. Thus, “percentage of sequence identity” or “percent identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. It would be understood that, when calculating sequence identity between a DNA sequence and an RNA sequence, T residues of the DNA sequence align with, and can be considered “identical” with, U residues of the RNA sequence. For purposes of determining percent complementarity of first and second polynucleotides, one can obtain this by determining (i) the percent identity between the first polynucleotide and the complement sequence of the second polynucleotide (or vice versa), for example, and/or (ii) the percentage of bases between the first and second polynucleotides that would create canonical Watson and Crick base pairs.

The Basic Local Alignment Search Tool (BLAST) algorithm, which is available online at the National Center for Biotechnology Information (NCBI) website, may be used, for example, to measure the percent identity between or among two or more of the polynucleotide sequences (BLASTN algorithm) or polypeptide sequences (BLASTP algorithm) disclosed herein. Alternatively, percent identity between sequences may be performed using a Clustal algorithm (e.g., ClustalW or ClustalV).

Standard sequence identity threshold values can identify fungi at different taxonomic levels, for example at the strain level, at the species level, or at the genera level. (See, for example, Sigisfredo et. al. 2016. FEMS Microbiology Ecology, 92:4, fiw045).

Hydrolytic and oxidative polypeptides. The ability of fungi to degrade material can be associated with enzymatic processes catalyzed by hydrolytic and oxidative polypeptides that cleave relevant linkages (e.g., ester, peptide, and C—C, bonds) in the polymers. (See, for example, Srikanth et al. Bioresour. Bio process. 9: 42).

Non-limiting examples of hydrolytic polypeptides include proteases and lipases. Proteases (also called a peptidases, proteinases, or proteolytic enzymes) are in a family of enzymes that catalyzes proteolysis, breaking down proteins into smaller polypeptides or single amino acids. Lipases are in a family of enzymes that break down triglycerides into free fatty acids and glycerol.

Non-limiting examples of oxidative polypeptides include laccases, peroxidases, and hydroxylases. Laccases are in a family of enzymes that catalyze oxidation reactions coupled to four-electron reduction of molecular oxygen to water. They can degrading lignin and can be found in many white-rot fungi. Peroxidases are in a family of enzymes that catalyze the oxidation of a substrate by hydrogen peroxide or an organic peroxide. Most peroxidases are ferric heme proteins. Hydrolases are in a family of enzymes that catalyze bond cleavages by reaction with water.

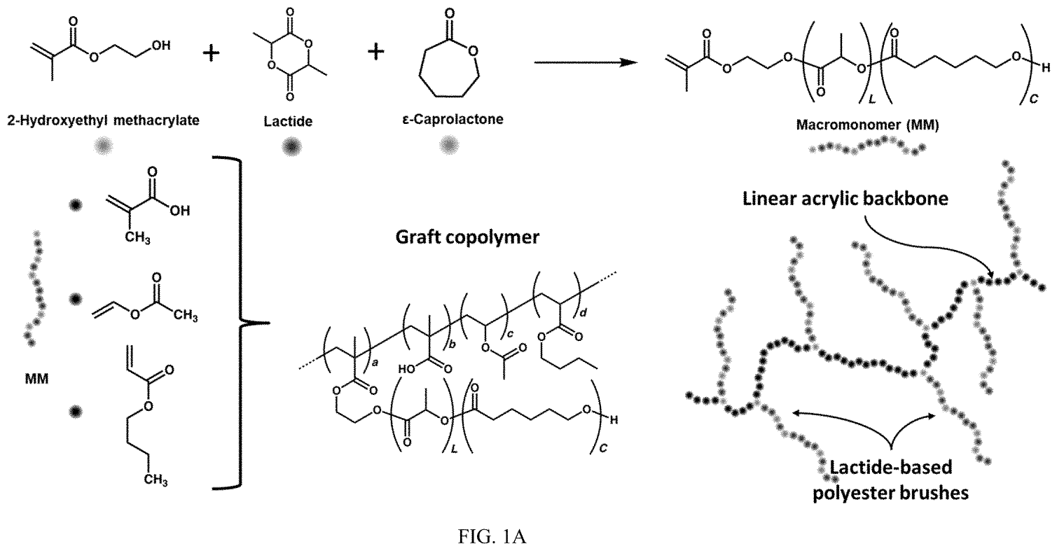

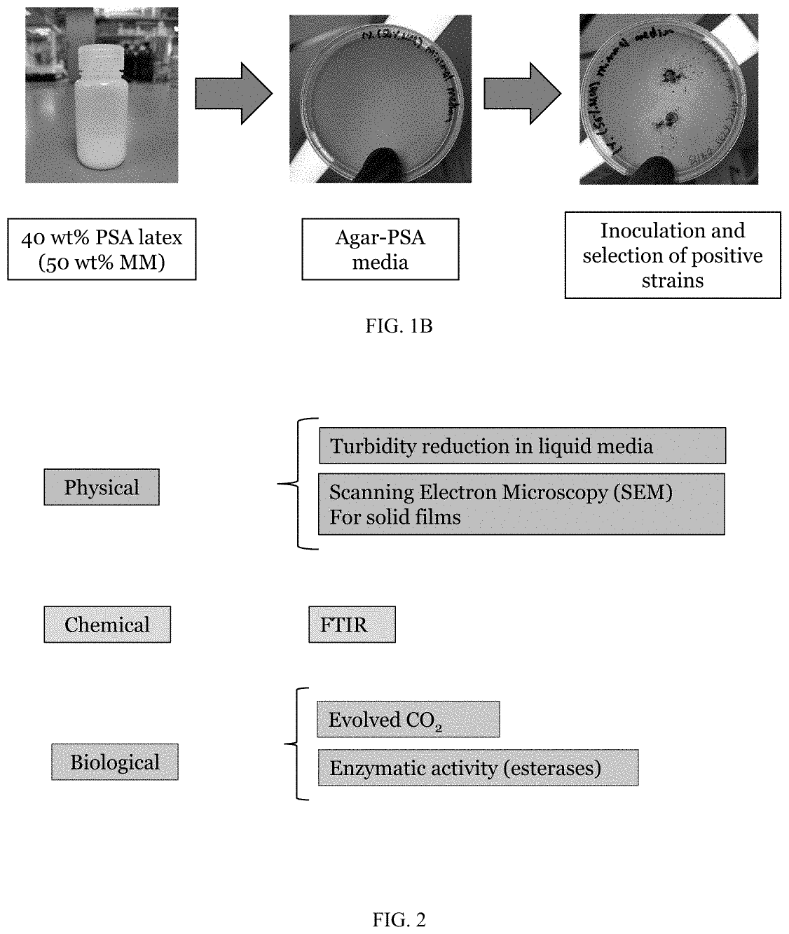

Polymers. In various embodiments, the polymer herein can be the reaction product of at least one monomer and at least one polyester graft. In some examples, the polymers described herein can include a carbon-carbon (C—C) backbone and a polyester graft (e.g., a macromonomer, also sometimes referred to as macromer) bonded (e.g., grafted) to the C—C backbone. The polymer described herein can include an acrylic polymer (or also referred to as an acrylate polymer). The polymer can be the polymerization product (or a reaction product) of at least an acrylate monomer. For example, the polymer can be the reaction product of at least a monomer selected from the group consisting of alkyl acrylates, alkyl methacrylates, and combinations thereof. The polymer can be or include the reaction product of at least one of an acrylate monomer, a lactone monomer (e.g., a lactide monomer or a caprolactone monomer), a styrene monomer, an alkyl vinyl ester monomer (e.g., a vinyl acetate monomer), and combinations thereof. For example, the C—C backbone of the polymer can include a linear acrylic backbone and lactide-based polyester brushes, as shown in FIG. 1A. Non-limiting examples of an acrylate monomer can include a (meth)acrylate monomer, a carboxylic acid functional (meth)acrylate monomer, a hydroxyl functional (meth)acrylate monomer, and a (meth)acrylamide monomer or its alkyl and hydroxy-alkyl derivatives.

The polymer can include one or more polyester grafts. In some embodiments, the polyester graft is a macromonomer. In some embodiments, the polymer is the reaction product of at least a lactide-based macromonomer, an acrylic-based macromonomer, or combinations thereof. In some cases, the polyester graft is the reaction product of a lactide monomer, an ε-caprolactone monomer, or combinations thereof. In some cases, the polyester graft includes the reaction product of a lactide monomer. In some embodiments, the polyester graft includes the reaction product of a monomer selected from the group consisting of alkyl acrylates, alkyl methacrylates, and combinations thereof. In some cases, the polyester graft includes the reaction product of other monomers, such as styrene monomers and/or vinyl acetate monomers.

In some cases, the polyester graft is grafted to the polymer (e.g., acrylic polymer). For example, in some cases, the reaction product of a lactide monomer, a caprolactone monomer, or a combination thereof, is grafted to the polymer. In some cases, the polymer is synthetically produced.

The polymer described herein can include a polymer comprising a polyester graft. In some cases, the polyester graft is a macromonomer. In some cases, the macromonomer is an acrylic functionalized polyester macromonomer. The acrylic functionalized polyester macromonomer can be copolymerized with acrylates in various embodiments. In some cases, the macromonomer is a reaction product of at least a lactide monomer, a caprolactone monomer, or combinations thereof. In some embodiments, the polymer is the polymerization product of at least a monomer selected from the group consisting of acrylates, methacrylates, and combinations thereof. In some embodiments, the polymer is the polymerization product of at least one monomer selected from the group consisting of alkyl acrylates, alkyl methacrylates, and combinations thereof and a macromonomer. In some cases, the polymer is the polymerization product of at least one monomer selected from the group consisting of alkyl acrylates, alkyl methacrylates, and combinations thereof and a lactide-based macromonomer, wherein the polymer comprises at least 15 wt. % of the lactide-based macromonomer. The lactide-based macromonomer may include reaction products of a lactide monomer. In some embodiments, the polymer is the polymerization product of at least an acrylate monomer and a macromonomer.

The amount of any of the macromonomers described herein that is included in any of the polymers described herein can be measured by percent weight (wt. %). For example, the polymer can include at least 0.1 wt. %. In some cases, the polymer can include at least 15 wt. % of a macromonomer (e.g., at least 20 wt. %, 25 wt. %, 30 wt. %, 35 wt. %, 40 wt. %, 45 wt. %, 50 wt. %, 55 wt. %, 60 wt. %, 65 wt. %, 70 wt. %, or 75 wt. % of a macromonomer). In some cases, the polymer can include a range of wt. % of a macromonomer. For example, the polymer can include from about 15 wt. % to about 50 wt. % of a macromonomer (e.g., from about 20 wt. % to about 50 wt. % macromonomer).

Non-limiting examples of polymers include pressure sensitive adhesives (PSAs). (See, for example, the detailed description of U.S. Pat. No. 9,469,797, which is incorporated in its entirety.) Briefly, polymers (e.g., PSAs) can be the polymerization product of: (A) a monomer selected from the group consisting of alkyl acrylates, alkyl methacrylates, and combinations thereof, and (B) a macromonomer comprising the reaction product of (a) a hydroxy-functional, ethylenically unsaturated monomer (e.g., a hydroxyalkyl acrylate or methacrylate where the alkyl group is a C—C alkyl group), (b) a bio-based monomer (e.g., a lactide), and (c) a modifying monomer. The modifying monomer may be a bio-based monomer, such as caprolactone. It lowers the Tg of the macromonomer relative to the same macromonomer prepared in the absence of the modifying monomer. The polymerization product preferably contains at least 25%, at least 35%, at least 40%, at least 50%, at least 55%, at least 60%, at least 70%, or at least 75% by weight of the macromonomer minus the hydroxy-functional, ethylenically unsaturated monomer, based upon the total weight of the polymerization product. In some cases, the polymer comprises at least 25% macromonomer. In some cases, the polymer comprises at least 30% of the macromonomer. In some cases, the polymer comprises at least 50% macromonomer. Suitable processes for preparing the polymerization product include aqueous-based polymerization processes such as emulsion and mini-emulsion polymerization processes.

Non-limiting examples of suitable (A) monomers include C—C alkyl acrylates (e.g., C1-C12 alkyl acrylates), alkyl methacrylates, and combinations thereof. Specific examples include n-butyl acrylate, n-butyl methacrylate, 2-ethylhexyl acrylate, and 2-ethylhexyl methacrylate.

Non-limiting examples of suitable hydroxyl alkyl acrylates and meth acrylates for the macromonomer include acrylates and meth acrylates capable of participating in a ring-opening polymerization of the lactide and lactone monomers. Specific examples include 2-hydroxy ethyl acrylate, 2-hydroxyethyl methacrylate, and combinations thereof. Examples of suitable lactides include D-lactide, L-lactide, and D,L-lactide.

Non-limiting examples of suitable lactones include ε-caprolactone.

In some cases, the polymer is a PSA. In some cases, the polymer is a water-based PSA (e.g., a latex product).

Additional additives. Any of the polymers described herein can also be combined with additional additives. For example, the polymers can be combined with any of the polymer-degrading fungal species described herein and with degradation-enhancing compounds to increase the rate of degradation.

Non-limiting examples of degradation-enhancing compounds can include organic-containing materials such as soil mixes, wood chips, wheat straw, and wheat bran. Soil mixes can contain topsoil, peat moss, vermiculite, or combinations thereof. In some cases, a soil mix includes topsoil, peat moss, and vermiculate in a 1:1:1 ratio. Non-limiting examples of wood chips include birch wood chips.

Composition formulations. Any of the compositions described herein can be provided, for example, as a liquid formulation, a powder formulation, or tablet formulation. Any of the formulations can contact (e.g., inoculate or be combined with) the polymer any number of appropriate times of a determined time frame (e.g., one time over a year, two times over a year, three times over a year, etc., or once daily, once weekly, once monthly, once every six months, etc.). Liquid formulations can be combined with any of the polymers described herein using any appropriate method (e.g., spraying, pouring, dripping) over any acceptable period of time.

A unit dosage of the formulations described herein can include at least 1 mg of the cells or spores of the polymer-degrading fungal species (e.g., about 5 mg, about 10 mg, about 100 mg, between about 1 mg and about 100 mg, between about 10 mg and about 1 g, between about 100 mg and about 10g).

Methods of Use.

Degrading polymers. Described herein are methods of degrading any of the polymers described herein. Methods can include contacting any of the polymers described herein with any of the polymer-degrading fungal species described herein, and degrading the polymer.

Also described herein are methods of contacting any of the polymers, any of the hydrolytic polypeptides, and any of the oxidative polypeptides; and degrading the polymer.

As used herein, “degrading” can include breaking any of the bonds in any of the polymers described herein to produce chemical components with smaller molecular weights, and can be measured using any standard methods (e.g., measuring weight loss of the polymer). Non-limiting examples of methods used to measure degradation can include weight loss of the polymer, SEM analysis, and FTIR analysis. Degrading can include a degradation of at least 5 wt. % of the polymer (e.g., between about 5 wt. % and about 95 wt. %). For example, degrading can include a degradation of at least 40 wt. % of the polymer (e.g., at least 50 wt. %, at least 60 wt. %, at least 75 wt. %, at least 80 wt. %, at least 85 wt. %, at least 90 wt. %, at least 95 wt. %, at least 97.5 wt. %, or at least 99 wt. % of the polymer) within a predetermined time period. Degrading can include a degradation from about 40 wt. % of the polymer to about 99 wt. % of the polymer (e.g., from about 50 wt. % to about 99 wt. %, from about 60 wt. % to about 99 wt. %, from about 75 wt. % to about 99 wt. %, from about 80 wt. % to about 99 wt. %, from about 85 wt. % to about 99 wt. %, from about 90 wt. % to about 99 wt. %, from about 95 wt. % to about 99 wt. %, from about 97.5 wt. % to about 99 wt. %, from about 40 wt. % to about 97.5 wt. %, from about 40 wt. % to about 95 wt. %, from about 40 wt. % to about 90 wt. %, from about 40 wt. % to about 85 wt. %, from about 40 wt. % to about 80 wt. %, from about 40 wt. % to about 75 wt. %, from about 40 wt. % to about 60 wt. %, from about 40 wt. % to about 50 wt. %, from about 45 wt. % to about 75 wt. %, or from about 50 wt. % to about 80 wt. %) within a predetermined time period.

Degrading any of the polymers described herein can occur within a predetermined time period of, for example, between about one day and one year. For example, degradation of the polymer occurs between about one day and one month or between about one week and six months. In some cases, the degrading occurs between about one day and one year. In some cases, the degrading occurs between about one week and six months. In some embodiments, degradation of at least 40% of the polymer (e.g., at least 50%, at least 60%, at least 75%, at least 80%, at least 85%, at least 90%, at least 95%, at least 97.5%, or at least 99% of the polymer) occurs within a predetermined time period of at least 1 day, 5 days, 10 days, 20 days, 1 month, 3 months, 6 months, and 1 year. In some embodiments, degradation of at least 40% of the polymer (e.g., at least 50%, at least 60%, at least 75%, at least 80%, at least 85%, at least 90%, at least 95%, at least 97.5%, or at least 99% of the polymer) occurs within a predetermined time period of from about 1 to about 5 days, about 1 to about 10 days, about 1 to about 20 days, about 1 to about 30 days, about 10 to about 30 days, about 15 to about 30 days, about 20 to about 30 days, about 1 to about 3 months, about 1 to about 6 months, about 2 to about 7 months, and about 6 months to about 1 year.

Degrading can occur in a controlled environment or an environment that is not controlled. In some cases, the degrading occurs in a controlled environment. Non-limiting examples of controlled environments include a partially closed container, a fully closed container, a partially closed container with a removable closure, and a fully closed container with a removable closure. Controlled environments can be found at, for example, waste management facilities, recycling facilities, commercial properties, and residential properties. In some cases, the degrading occurs in an environment that is not controlled. Non-limiting examples of an environment that is not controlled can include environments that are exposed to the weather (e.g., a recycling or waste management pile or dumpster).

In some cases, the degrading occurs in an environment where the polymers have been enriched. For example, the degrading can occur in an environment (e.g., a controlled environment or an environment that is not controlled) that contains other materials in an amount less than the amount of the polymer. In some cases, the degrading can occur in an environment (e.g., a controlled environment or an environment that is not controlled) that contains other materials in an amount greater than the amount of the polymer. In some cases, the environment (e.g., a controlled environment or an environment that is not controlled) does not contain significant amounts of other carbon-containing materials. As used herein, “carbon-containing materials” are materials that exclude the polymer.

Contacting any of the polymers described herein with any of the one or more polymer-degrading fungal species described herein, or with any of the hydrolytic polypeptides, and any of the oxidative polypeptides, can include mixing the polymers with the polymer-degrading fungal species or with the polypeptides. In some cases, the step of contacting can also include combining any of the additional additives described herein (e.g., degradation-enhancing compounds, for example, wheat bran) with the polymer and fungal species.

Methods of assessing polymer degradation. Polymer degradation assays can assess a polymer-degrading fungal species' ability to degrade a polymer, including any of the polymers described herein. Non-limiting examples of polymer degradation assays include a drop assay (also called an agar plate screening assay), a liquid degradation assay, a weight loss assay, and a growth-on-polymer assay. Other assays can include genomic analysis to identify the presence of genes involved in polymer degradation (e.g., the identification of the presence of any of the hydrolytic polypeptides described herein and any of the oxidative polypeptides described herein).

A drop assay places a drop of a liquid fungal culture, a streaked fungal colony, or fungal spores on a solid agar plate (test location) that contains the polymer as the main or sole carbon source. The presence of the polymer is detectable or measurable, either by eye (e.g., an opaque polymer) or by another imaging technique for an agar plate or agar derived from an agar plate (e.g., fluorescence, bioluminescence, a colorimetric assay, etc). After the fungal species is placed on the agar plate, the plate is incubated to encourage fungal growth, if possible. After an appropriate amount of time, the agar plates are analyzed to determine if the polymer is no longer present (or a smaller amount of polymer is present) in and around the areas in which the fungal strain was inoculated. If the polymer is an opaque polymer when in an agar plate, polymer degradation can be viewed as a clearing or halo around the test location.

A liquid degradation assay places a liquid fungal culture, a fungal colony, or fungal spores in a liquid medium that contains the polymer as the main or sole carbon source. The presence of the polymer is detectable or measurable. For example, a specific absorbance wavelength (e.g., absorbance of 590-600 nm, 595 nm) can detect the presence of the polymer, or by another imaging technique for a liquid culture (e.g., fluorescence, bioluminescence, a colorimetric assay, etc). After the liquid culture is inoculated with the fungal species, the liquid culture is incubated to encourage fungal growth. After an appropriate amount of time, the liquid culture is analyzed to determine if the amount of the polymer (either absolute or a relative amount of the polymer) decreased during incubation with the fungal species. A reduction in the amount of the polymer indicates that the fungal species degraded the polymer and is a polymer-degrading fungal species.

A weight loss assay places a liquid fungal culture, a fungal colony, or fungal spores on a solid polymer film that was weighed (initial weight). After the film is inoculated with the fungal species, the solid polymer film is incubated to encourage fungal growth. This can include incubating the solid polymer film and fungal species in the presence of any of the additional additives described herein. After an appropriate amount of time, the solid polymer film is removed from the incubation environment, is washed off, and is dried. The weight of the solid polymer film is measured (final weight) and the difference between the initial weight and the final weight is determined. A reduction in the weight of the solid polymer film indicates that the fungal species degraded the polymer and is a polymer-degrading fungal species.

A growth-on-polymer assay places a liquid fungal culture, a fungal colony, or fungal spores on a solid polymer film or in a liquid medium that contains the polymer as the main or sole carbon source. After the solid polymer film or liquid medium is inoculated with the fungal species, the number of inoculated cells is determined using any standard technique (e.g., plating to count colonies, absorbance measurement, turbidity measurement, etc), and the inoculated solid polymer film or liquid culture is incubated to encourage fungal growth. After an appropriate amount of time, the number of fungal cells is determined through any standard technique. More fungal cells after incubation compared to the number cells inoculated indicates that the fungal species grew on the polymer, and therefore the polymer was degraded to free nutrients for the fungal species to grow. As such, fungal species that can grow on a polymer can be fungal species that degrade the polymer and are polymer-degrading fungal species.

### EXAMPLES

### Example 1. Fungal Degradation Behavior of a High-Biomass Content, Mixed Pressure-Sensitive Adhesive

To determine if fungi could degrade the ester linkages present in bio-based PSAs that also included a linear acrylic backbone and lactide-based polyester brushes (FIG. 1A), a series of assays were used to identify fungal candidates.

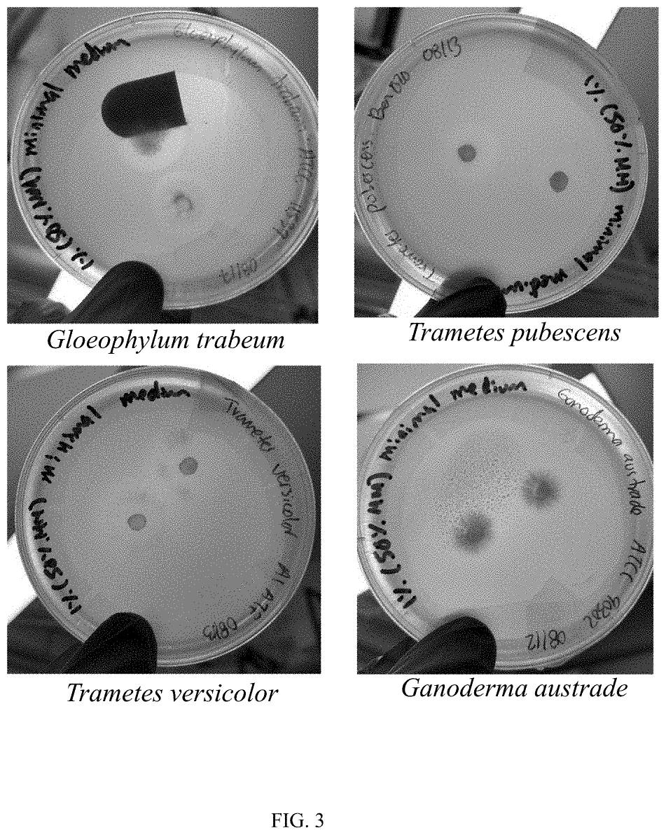

Fifty fungal candidates that were able to degrade PSAs were identified (FIG. 1B). Briefly, a semi-opaque medium of 40% wt/wt PSA-latex with 50% MM was plated to generate an agar-PSA medium. A variety of fungal strains were inoculated on the plates and incubated. A zone of clearing around the area of inoculation (i.e., being able to see through the otherwise semi-opaque agar) indicated that the fungal species were able to degrade the agar-PSA. FIG. 3 shows exemplary images of the zones of clearing. From 53 fungi evaluated, 23 generated clearance zones in the PSA-agar media and were identified as positive strains. The 23 strains included T. versicolor, H. grisea, A pullulans, R. bicolor, F. hepatica, F. mediterranea, T. hirsuta, L. betulina, P. stipticus, P. crispa, G. austrade, G. trabeum, A. niger, L. edodes, L. Mollusca, P. chamaeropsis, C. puteana, G. lucidum. T. pubescens, Penicillum sp. Wall, P. microspora, L. olivascens, and Armillaria sp.

After identification of a variety of fungal species that may degrade PSA, additional PSA degradation experiments were conducted. A schematic depicting an overview of a variety of different experiments and techniques that could be used to measure PSA fungal biodegradation is shown in FIG. 2. Optional experiments and techniques include physical experiments to assay the turbidity reduction (i.e. degradation) in liquid medium and scanning electron microscopy (SEM) to assay degradation of solid films containing PSA, chemical experiments such as Fourier Transform Infrared Spectroscopy (FTIR), and biological experiments to assay evolved, or produced, CO₂ and enzymatic activity of, for example, esterases.

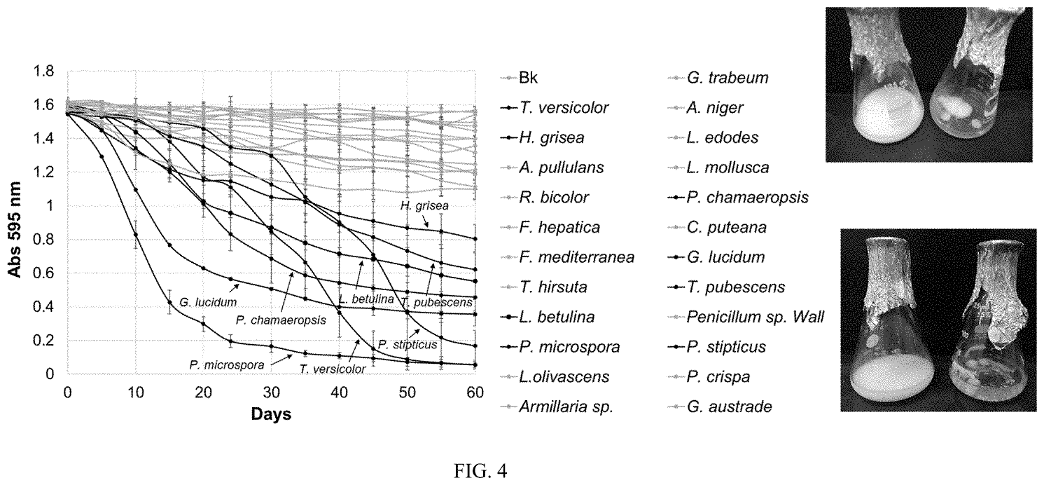

To measure PSA degradation by the 23 positive strains, a turbidity reduction assay was used. The positive strains were incubated with a liquid PSA-latex containing medium over a period of 60 days. The absorbance at 595 nm (Abs 595 nm) was measured periodically to generate a turbidity reduction curve (FIG. 4). The top PSA degraders were P. microspora, T. versicolor, P. stipticus, G. lucidum, P. chamaeropsis, L. betulina, T. pubescens, and H. grisea.

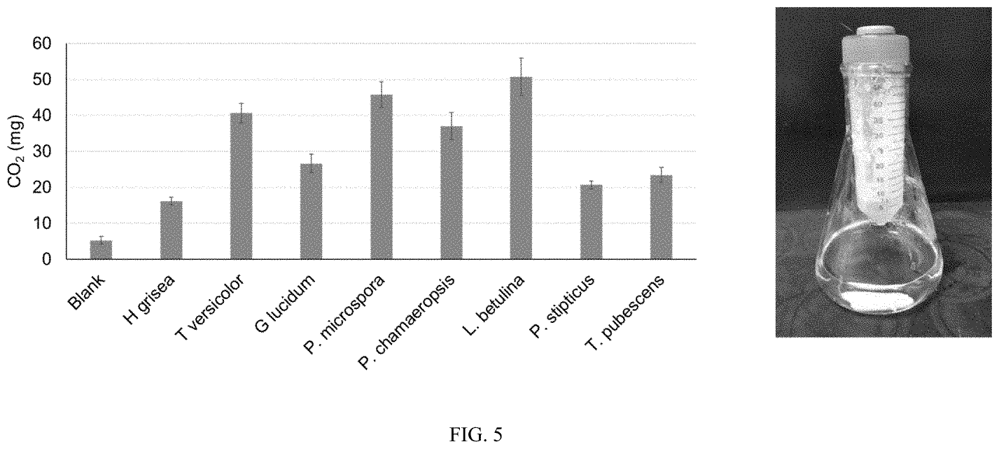

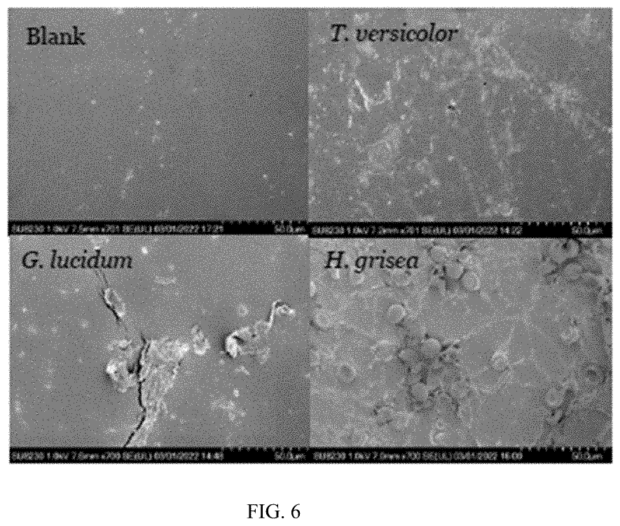

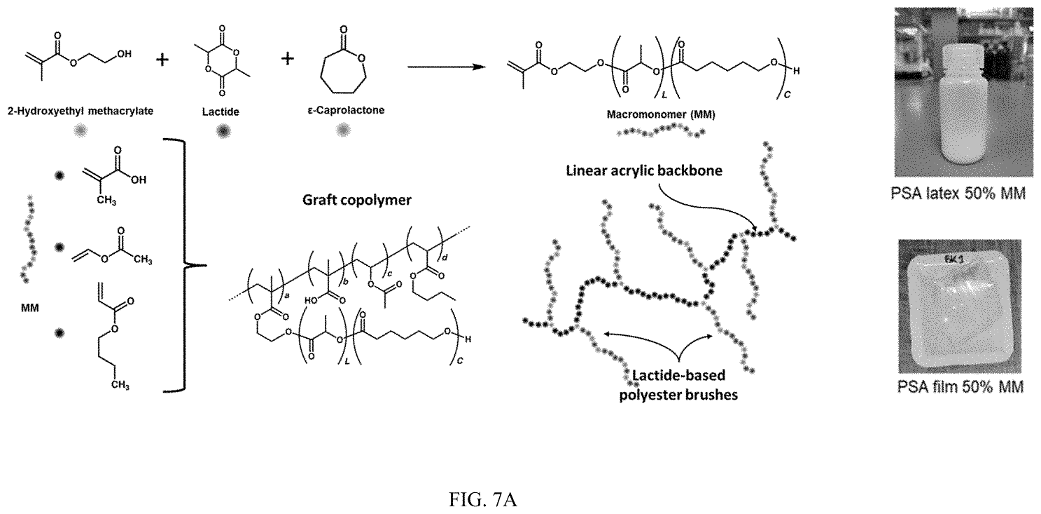

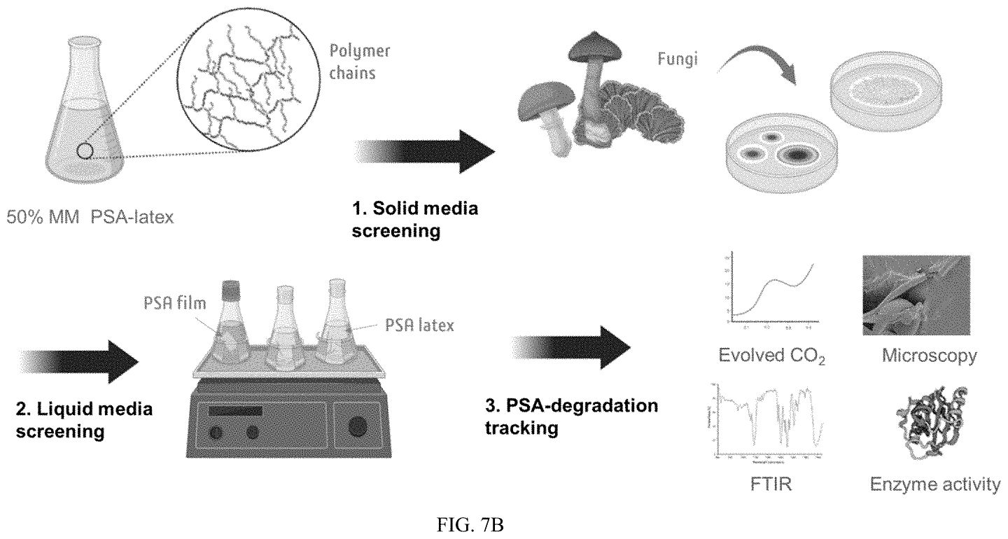

CO₂ evolution from a PSA-latex containing medium when incubated with fungal species indicates fungal growth using the PSA-latex as the sole carbon source and therefore degradation of the PSA-latex. CO₂ evolution, or production, was measured for 8 fungal species over a period of 30 days (FIG. 5).

Weight loss from a PSA film, indicating degradation, was observed for the fungi G. lucidum, T. versicolor, and H. grisea after 90 days of incubation of the PSA film as the sole carbon source (Table 1). SEM images of the PSA films incubated with fungi for 90 days illustrate fungal growth and degradation as well (FIG. 6).

TABLE 1
Weight Loss of PSA film

| .Fungal Species | Weight loss (%) |
|---|---|
| P. microspora | 22.36 ± 0.72 |
| P. chamaeropsis | 22.31 ± 1.71 |
| G. lucidum | 18.73 ± 1.29 |
| L. betulina | 17.53 ± 0.77 |
| T. versicolor | 16.89 ± 1.38 |
| H. grisea | 11.96 ± 1.00 |
| P. stipticus | 11.74 ± 0.13 |
| Blank (control) | 9.11 ± 0.02 |

### Example 2. Fungal Biodegradation of Hybrid Adhesive Polymers Containing High-Biomass Contents

This example reports on the fungal degradation behavior of hybrid PSAs copolymerized mainly from traditional acrylics and lactide-based macromonomers (MMs). These structures provide high performance at a reasonable cost and represent an alternative approach to addressing environmental concerns. Evaluating the biodegradability of these hybrid PSAs involved solid and liquid cultures using 53 unique fungal species (See Table 2.). Nearly 50% (n=23) of the screened species showed PSA-degrading capacities of varying degrees, including Trametes versicolor and Pestalotiopsis microspora, providing roughly complete (>96%) polymer removal in liquid cultures. Enzyme assays, namely laccases, lipases, and proteases, showed a high correlation with the observed biodegradation rates, suggesting that hydrolytic and oxidative enzymes were likely factors driving latex degradation. Fungal degradation of cast adhesive films was monitored using evolved CO₂ and mass loss and confirmed through scanning electron microscopy (SEM) and spectral analysis (FTIR). These results demonstrate that the high-performing hybrid formulation is degradable by fungi and could serve as a bridge technology, helping to usher in more sustainable disposable consumer products.

TABLE 2
Fungal Species Screened for PSA degradation Capacity.

| | | Halo |
|---|---|---|
| | Wood | (Yes indicates |
| Fungus | rot type | degradation) |
| Leucogyrophana mollusca ATCC | Not | No |
| 38822 | determined | |
| Lasiodiplodie missouriana | N/A | No |
| Fusarium sp. (like solani) | N/A | No |
| Pestalotiopsis chamaeropsis | N/A | Yes |
| Ganoderma lucidum ″stramets″ | White rot | Yes |
| Gloeophylum trabeum ATCC 11539 | Brown rot | Yes |
| Stereum hirsutum FP91666 | White rot | No |
| Lenzites betulina ATF03 | White rot | Yes |
| Trametes pubescens Ban020 | White rot | Yes |
| Penicillum ochrochlorum ATCC 9112 | N/A | No |
| Trametes versicolor A1 ATF | White rot | Yes |
| Pleurotus ostreatus ATCC 32783 | White rot | No |
| Alternaria sp. | Not | No |
| | determined | |
| Coprinellus micaceus FP 101792 | N/A | No |
| Wolfiporia cocos MD104: 5510 | Brown rot | No |
| Leucogyrophana olivascens ATCC | Not | Yes |
| 22108 | determined | |
| Lasiodiplodia crassipora | N/A | No |
| Plicaturopsis crispa FD-325.553 | White rot | Yes |
| Fistulina hepatica ATCC 64428 | Brown rot | Yes |
| Armillaria sp. | White rot | Yes |
| Lentinus edodes ATCC 48883 | White rot | Yes |
| Humicola grisea ATCC 16298 | Soft rot | Yes |
| Aurebasidium pullulans ATCC 9348 | Soft rot | Yes |
| Aspergillus niger ATCC 6275 | Soft rot | Yes |
| Penicillum sp. Wall BD | Soft rot | Yes |
| Trichoderma viride ATCC 32630 | Soft rot | No |
| Aspergillus oryzae ATCC 14893 | Soft rot | No |
| Coniophora puteana ATCC 12675 | Brown rot | Yes |
| Merulliporia incrassata | Brown rot | No |
| Antrodia vaillantii ATCC 11044 | Brown rot | No |
| Dichomitus squalens Het-26-1 | White rot | No |
| Neolentinus lepideus | Brown rot | No |
| Phlebia tremallosa TAB78 | White rot | No |
| Piptoporus betulinus A5ATF | Brown rot | No |
| Irpex lacteus ATCC 11245 | White rot | No |
| Ceriporiopsis subvermispora ATCC | White rot | No |
| 90467 | | |
| Resinicium bicolor ATCC 64897 | White rot | Yes |
| Panellus stipticus | White rot | Yes |
| Serpula himantiades ATCC 36335 | Brown rot | No |
| Neurospora crassa | Soft rot | No |
| Trichoderma reesei | Soft rot | No |
| Fomitopsis pinicola FP 10587718 | Brown rot | No |
| Fomitopsis lilacinogilva ATCC 46156 | Brown rot | No |
| Fomitopsis cajenderi ATF 04 | Brown rot | No |
| Fomes fomentarius ATF | White rot | No |
| Trametes hirsuta ATCC 34679 | White rot | Yes |
| Phlebiopsis gigantea NF 2-2-B | White rot | No |
| Schizophylum commune ATCC 4819 | Grey rot | No |
| Ganoderma austrade ATCC 90302 | White rot | Yes |
| Coniophora arida ATCC 56488-T | Brown rot | No |
| Fomitiporia mediterranea MF | White rot | Yes |
| Pestalotiopsis neglecta | N/A | No |
| Pestalotiopsis micropsora | N/A | Yes |

Methods

Biobased macromonomer and PSA synthesis. The MM and hybrid PSA syntheses are described elsewhere. (See, for example, Pu et al. Macromol. React. Eng. 7: 515-526). Briefly, the MM was produced via the bulk ring-opening polymerization of L-lactide and ε-caprolactone using 2-hydroxyethyl methacrylate as an initiating head-group in a 5:4:1 molar ratio, respectively. Stannous octoate was used as a catalyst to start the polymerization at 0.15 mol % (based on moles of total monomer). Reactions ran for 24 hours in an oil bath (140° C.) and were terminated by allowing products to cool to room temperature. The resulting MMs were used without further purification.

PSAs were synthesized using miniemulsion polymerizations of MMs with monomers commonly used to generate PSAs, including butyl acrylate, methacrylic acid, and vinyl acetate. Hybrid polymers containing various levels of MM were generated, ranging from 0 to 70 wt % MM, but most of the studies utilized an adhesive having an equal mass mixture (50:50) of traditional monomers and MMs. The monomers were dispersed in water using a combination of anionic and nonionic nonylphenol ethoxylate surfactants at 3.5 wt % actives based on total monomer weight. The total solids of formulated emulsions were approximately 40 wt %. Miniemulsions were reacted to form adhesive latexes by heating to 80° C. in an oil bath, adding a potassium persulfate initiator and maintaining reaction conditions for three hours.

PSA films were generated by weighing approximately 1.5 grams (g) of liquid latex into polystyrene weighing boats, followed by drying overnight in an oven set to 80° C.

Adhesive-agar plate screening (Drop assay). To test the fungal ability to degrade the hybrid adhesive, a 1% PSA-agar plate was prepared with the following composition: 2.622 grams per liter (g/L) NaH₂PO₄·H₂O, 1.219 g/L K₂HPO₄, 0.086 g/L (NH₄)₂SO₄, 0.735 g/L HOC(COONa)(CH₂COONa)₂·2H₂O (sodium citrate), 0.123 g/L MgSO₄·7H₂O, 0.500 g/L NH₄NO₃, 0.015 g/L CaCl₂·2H₂O, 0.05 mM FeCl₃, 0.014 mM ZnCl₂, 0.012 mM CoCl₂·6H₂O, 0.21 μM Na₂MoO₄·H₂O, 1 μM thiamine, 0.011 mM CuCl₂·2H₂O, 0.012 mM MnCl₂·4H₂O, 0.012 mM H₃BO₃, and 15 g/L agar. The mineral solution was adjusted to a pH of 5.5, mixed with agar, autoclaved at 121° C. for 20 minutes, and then cooled to 60° C. before adding 25 milliliters (mL) of the 40% solids content PSA latex under sterile conditions. The media was agitated briefly and poured into Petri dishes. Fifty-three fungal species (Table 2) were inoculated on this solid media. The plates were incubated at 28° C. for 1-3 weeks. The appearance of a clearance halo around the growing colonies indicated degradation, and was scored as a positive result. Inoculated plates were used as blanks.

Liquid culture screening of latex samples (Liquid degradation assay). To quantitatively measure the PSA-degradation rate of the screened fungal degraders, a liquid medium was prepared with the same composition as the adhesive-agar plate medium as described above. 120 mL Erlenmeyer flasks containing 30 mL of media were inoculated with three 5 millimeter (mm) agar discs taken from the edge of fungal colonies grown in potato dextrose agar. The flasks were incubated at 28° C. and 115 rpm for 60 days. During this period, 400 μL samples were withdrawn every five days under sterile conditions, and turbidity was measured using absorbance (Abs) at 595 nm on a microplate reader (Bio-Rad iMark 1.02.01, USA). The degradation rate was calculated using Equation 1. Three biological replicates were analyzed for each fungus. Digital images were also taken regularly to visualize any clearance in the samples compared to the non-inoculated blank.

$$Degradation rate ( \% ) = \frac{{Abs}_{blank} - {Abs}_{sample}}{{Abs}_{blank}} \qquad [ 1 ]$$

Characterizing adhesive film degradation. Adhesive films were degraded in 120 mL Erlenmeyer flasks containing 30 mL of liquid media, with fungi that showed high degradation abilities (>50%) according to the liquid culture screening test described above. The species evaluated included Trametes versicolor, Pestalotiopsis microspora, Ganoderma lucidum, Humicola grisea, Pestalotiopsis chamaeropsis, Trametes pubescens, Lenzites betulina, and Panellus stipticus. The cultures were shaken at 28° C. and 115 rpm for 90 days. Following this degradation period, films were recovered from the liquid media, thoroughly washed to remove fungal hyphae, and dried at 60° C. for 24 h. For the scanning electron microscopy (SEM) analysis, the degraded film samples was coated with a 5 nm platinum layer using Leica EM ACE600 High Vacuum Sputter Coater (USA), and SEM images were collected using a Hitachi SU8230 Field Emission Gun (Japan). Film samples were also analyzed by Fourier Transform Infrared Spectroscopy (FTIR) using attenuated total reflectance (ATR). Spectra were obtained with Nicolet™ iS50 FTIR Spectrometer (USA), scanning from 400 to 4000 cm⁻¹ at room temperature.

Fungal respiratory activity. To test the ability of the fungi to use adhesive films as a sole carbon source, the fungi were cultured in the liquid media described previously without sodium citrate. The fungal strains were inoculated in 250 mL flasks containing 80 mL of media and a piece of solid adhesive film. Additionally, flask openings were fitted with 50-mL Falcon tubes with holes for air exchange. The Falcon tubes were filled with 12 mL of 0.05 M Ba(OH)₂. Ba(OH)₂ solutions were recovered and replenished every 5-6 days. The Ba(OH)₂ reacted with CO₂ to form BaCO₃ (Equation 2), which can be titrated with HCl (0.05 M solution) to determine the amount of CO₂ released due to film degradation (Equation 3).

Ba(OH)₂+CO₂→BaCO₃+H₂O [2]

Ba(OH)₂+2HCl→BaCl₂+2H₂O [3]

Weight loss assay. While assaying the fungal respiratory activity, the weight of the solid adhesive films were also monitored by weighing dried recovered films at 60° C. for 24 h to determine film degradation.

Enzyme activity evaluation. Laccase activity was measured with the substrate ABTS (2,2′-azino-bis (3-ethylbenzothiazoline-6-sulphonic acid). See, for example, Castaño et al. Biocatal. Agric. Biotechnol. 4: 710-716. Briefly, 170 μL of 50 mM acetate buffer pH 5.0 were mixed with 10 μL of 10 mM ABTS in a 0.2-mL PCR tube. The reaction was started by adding 20 μL of supernatant, briefly centrifuged at 5000 rpm for 10 s. The oxidation of ABTS was monitored at a wavelength of 415 nm, and the activity was determined using the molar extinction coefficient of ABTS (ε=36,000 M⁻¹cm⁻¹) as the amounts of μmoles of ABTS oxidized per minute under the assay conditions.

Protease activity was measured. See, for example, Liu et al. Prey. Nutr. food Sci. 18: 273-279. Briefly, 65 μL of 1% casein (prepared in 0.05 M phosphate buffer pH 7.0) was mixed with 65 μL of crude enzyme extract in a 0.2 mL PCR tube. The samples were incubated for 120 minutes at 45° C. in a thermocycler. Subsequently, the enzyme mixture was added to 390 μL of 10% trichloroacetic acid in a 1.5-mL microcentrifuge tube. The tubes were incubated for 15 minutes at RT with constant shaking, then centrifuged at 10,000 rpm for 10 minutes. Carefully, without disturbing the pellet, 120 μL of supernatant were taken from the tubes and added to 120 μL of 1 M NaOH. The absorbance at 450 nm was recorded. The substrate blank contained 65 μL of 1% azocasein and 65 μL of 0.05 M phosphate buffer pH 7. The enzyme blank consisted of 65 μL of crude extract and 65 μL of 0.05 M phosphate buffer pH 7. One unit of enzyme activity was defined as the increase in 0.01 absorbance units per minute with respect to the substrate blank.

Esterase and lipase activity were measured with 4-nitrophenyl-butyrate and 4-nitrophenyl-palmitate, respectively. See, for example, Kumar et al. Protein Expr. Purif. 41: 38-44. Briefly, 100 μL of 50 mM phosphate buffer pH 7, 30 μL of the substrate (5 mM 4-nitrophenyl-butyrate or 3.5 mM 4-nitrophenyl-palmitate), and 30 μL of enzyme extract were mixed in a 0.2 mL PCR tube. The tubes were incubated at 40° C. for 1 h, and 120 μL from this mixture were added to 80 μL of 0.5% Na₂CO₃. The absorbance of the samples at 415 nm was then recorded. One unit of enzyme activity was defined as the amount of p-nitrophenol released per minute under the assay conditions.

Heatmaps with enzyme activity and PSA clearance results from the liquid culture were obtained using the online tool heatmapper (heatmapper.ca). The correlation matrix was obtained using the software R 4.0.2 and the package Hmisc. The enzyme activity results were used to build a multiple regression model using the same software.

Results

Fungal degradation test results for hybrid copolymers generated via emulsion polymerization with traditional, primarily acrylic, monomers and various lactide-based macromonomers (MMs) concentrations are summarized below. These reactions generated stable latexes, which were coated to form adhesive films suitable for performance testing. The typical formulation contains 50 wt % MM. Films cast from this formulation and tested using ASTM standard tack, peel, and shear measurements show performance levels that meet or exceed those required for permanent tape, label, and packaging applications. In addition to the most commonly studied formulation, a series of hybrid PSAs containing MM levels ranging from 0 wt % to 70 wt % were generated and used to determine the influence of the lactide-based MMs on the rate and extent of the fungal degradations.

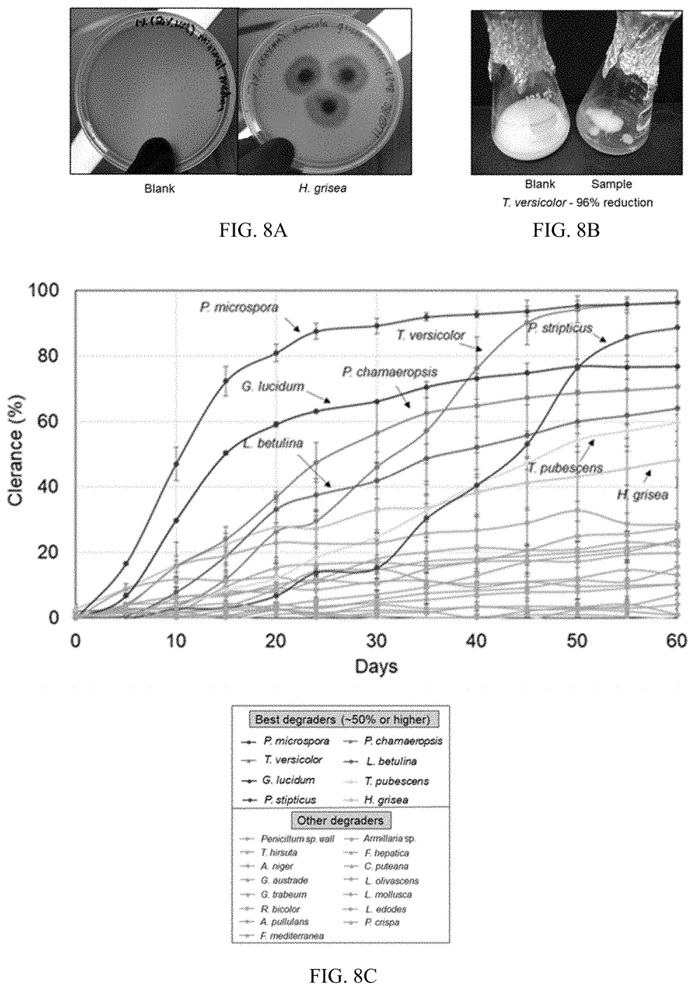

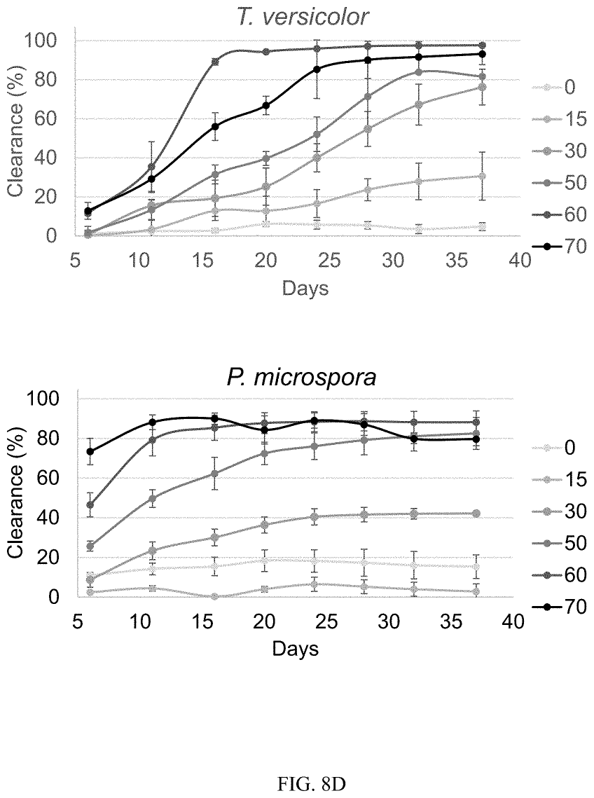

High-throughput screening. Initial studies involved a rapid plate screening method described in the Methods section. This test was used to identify fungal species that degraded the hybrid adhesive formula (FIG. 8A). This test also allowed identification of more active species from the extent of the clearance halos formed, indicating more significant fungal degradation. 23 out of 53 fungi evaluated (43%) showed degradation halos (FIG. 8A). A complete list of these fungi and their screening results are available in Table 2.

To more quantitatively evaluate the fungal species' PSA-degrading abilities, degradation tests in liquid cultures were performed, which tracked the progress of the degradation by monitoring changes in turbidity (Abs 595 nm). FIG. 8C shows that most of the 23 fungi could clear the media to some extent, and among these, eight notable degraders removed 50% of the hybrid PSA in 60 days. Remarkably, T. versicolor and P. microspora removed almost 100% of PSA within 50 days, evident by the high media transparency compared to the blank (FIG. 8B), with P. microspora removing>70% of substrate in only two weeks The effects of MM concentrations on degradation rate using P. microspora and T. versicolor were also investigated (FIG. 8D). The results showed that higher MM contents could enhance the adhesive clearance rate. Both fungi can achieve more than 80% PSA removal within 20 days when more than 50 wt % MM is used to generate the adhesive, with higher MM weight percentages producing increased removal rates. Notably, P. microspora reached>70% degradation after only five days of incubation for the formulations containing 70 wt % MM.

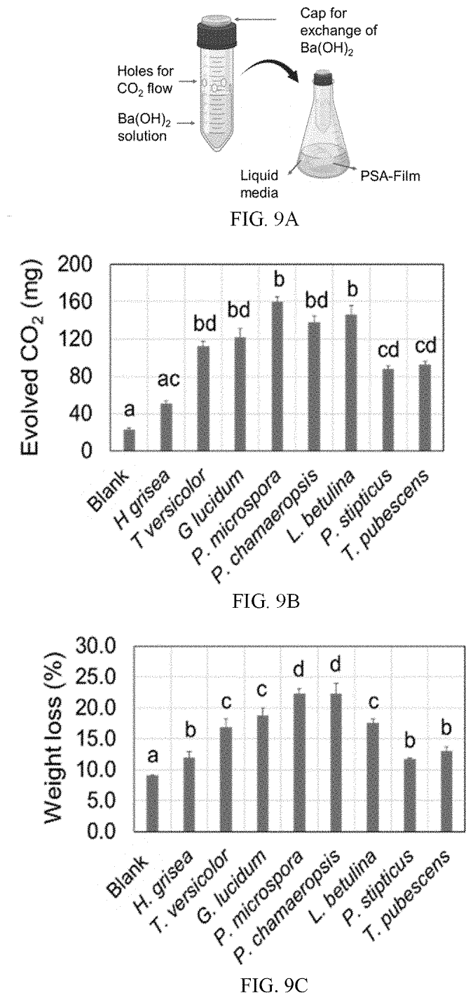

Respirometry test results. To evaluate the ability of fungi to utilize the hybrid PSA formula as a sole carbon source, respirometry tests were conducted. These involved growing fungi on adhesive films in a minimal media without sodium citrate (FIG. 9A). The measurements showed that the evolved CO₂ was significantly higher in the fungal cultures relative to the blank (ANOVA test, p<0.05) (FIG. 9B), except for H. grisea. P. microspora showed the highest level of evolved CO₂ (160 mg/day) among the eight screened fungi. In general, this aligned with the latex removal rates presented in FIG. 8C. Also, the CO₂ respiration rate was in agreement with the significant mass loss of the PSA films in the fungal treatments, which indicated that fungi could convert the adhesive carbon into CO₂. Among these, P. microspora caused the most mass loss (23%).

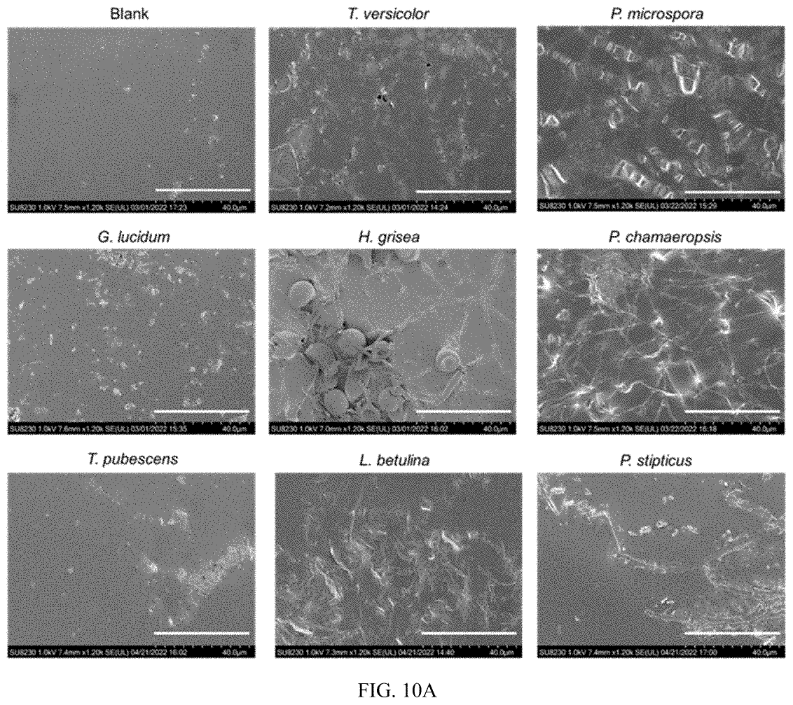

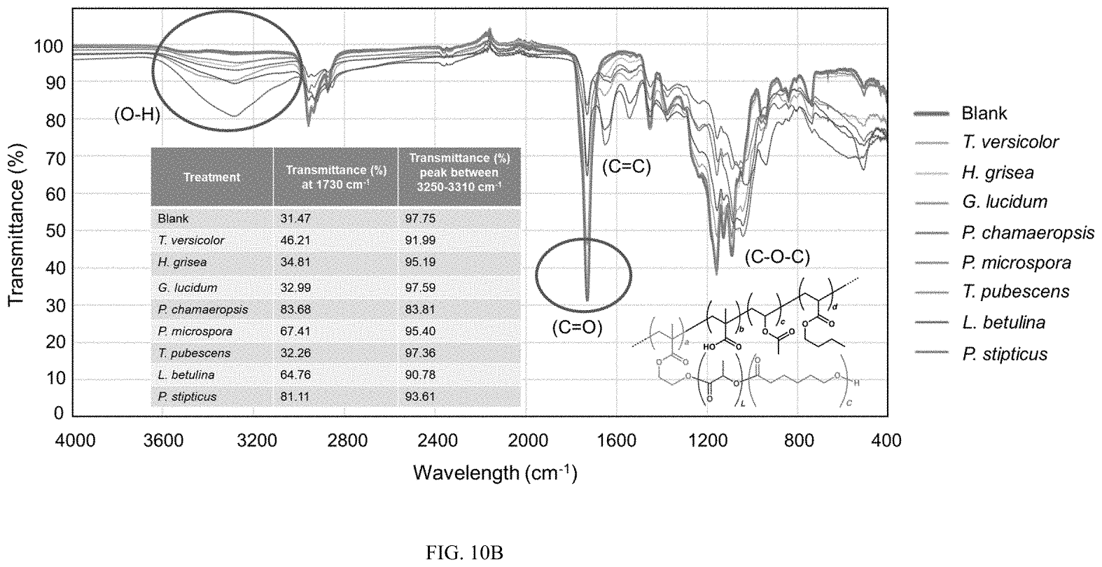

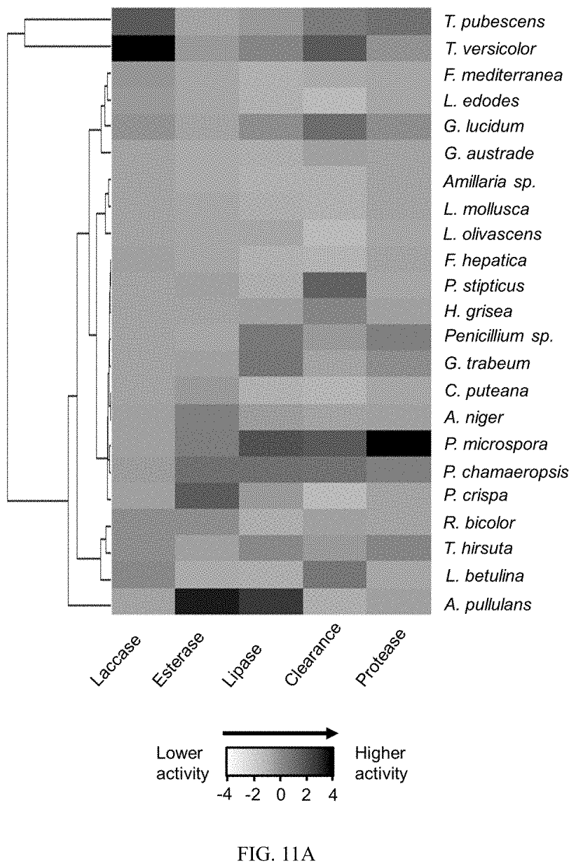

Evaluating structural changes in adhesive films due to fungal degradation. After confirming the fungal abilities to degrade the hybrid PSA films, the films were further characterized using SEM and FTIR spectroscopy. After 90 days of degradation, SEM images showed abundant fungal growth on the film surfaces and some disintegration compared to the smooth surface found for the non-degraded film samples (FIG. 10A). Some species, such as P. microspora, P. chamaeropsis, and H. grisea showed abundant surface changes, suggesting their hyphal cells might degrade the PSA film through a direct attachment.

FTIR analysis revealed changes in the functional groups present at the surface of the PSA films after the fungal treatment. There was an apparent decrease in carbonyl groups (C=O) compared to the blank, as well as an increase of the OH-stretching band associated with carboxylic acids (FIG. 10B). This likely results from the loss of ester groups due to the action of esterases, lipases, and other hydrolases followed by the mineralization to CO₂. In addition, other fungal oxidative enzymes such as laccases or monooxygenases may also cause the increase of carboxylic acid groups in the PSA film. Changes related to the C=C and C—O—C bonds were also found after fungal degradation, although they were more difficult to assign to specific changes occurring during biodegradation.

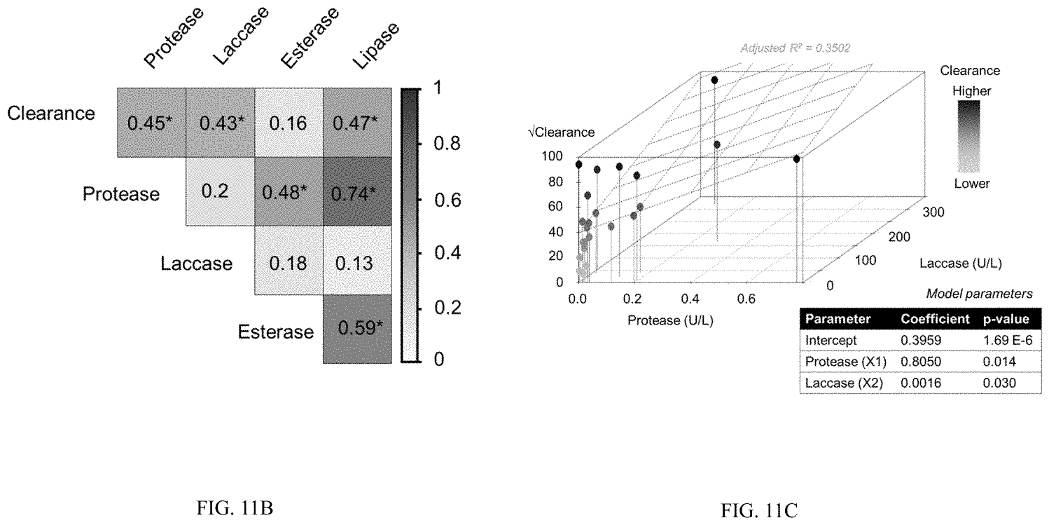

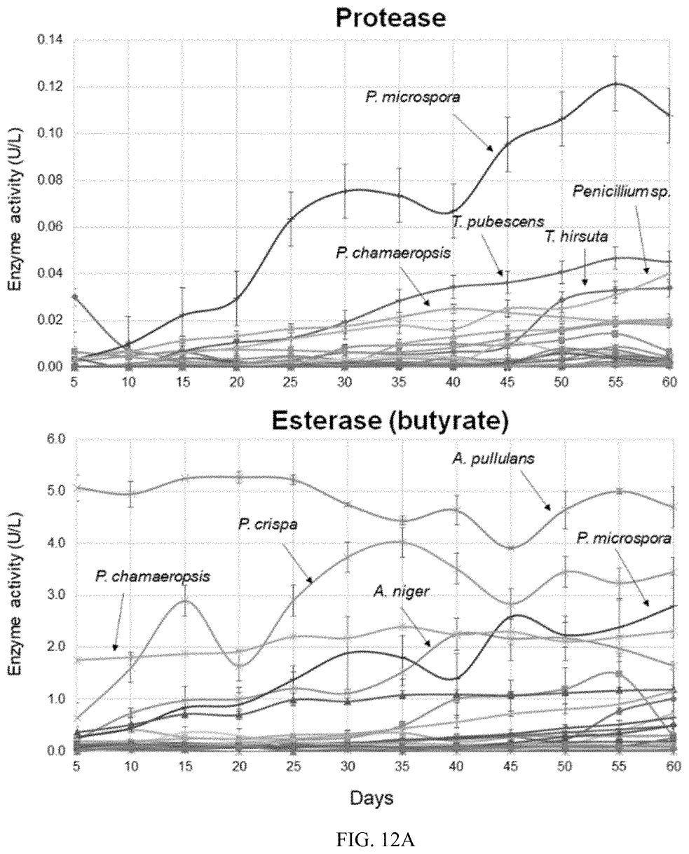

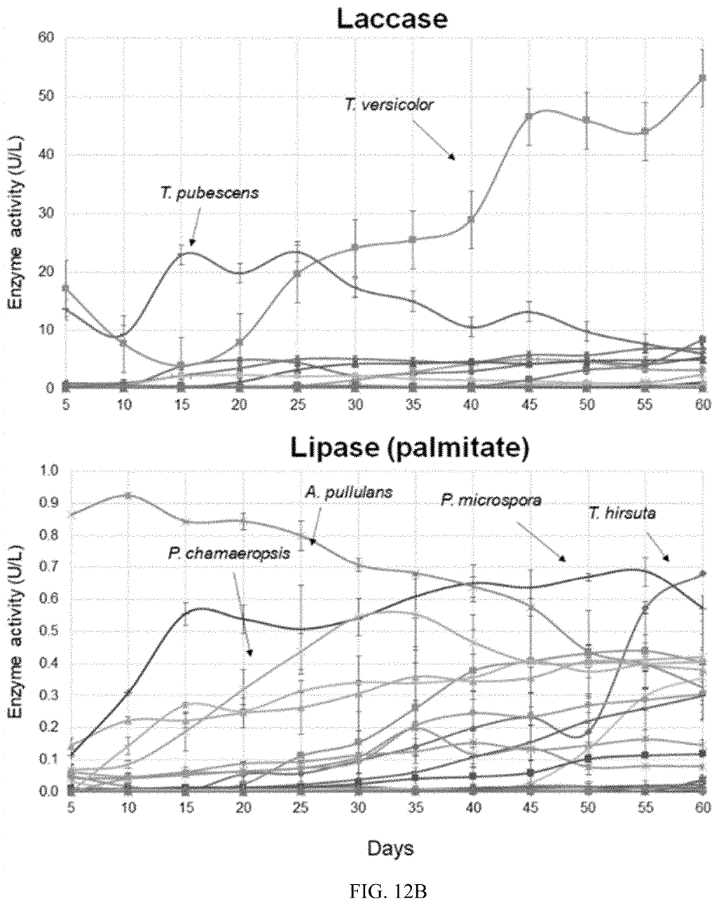

Identifying the presence of enzyme activity. The mechanisms underlying the fungal biodegradation of the hybrid adhesive were further investigated with a genome-wide analysis. The correlation between adhesive removal rates and 14 degradative enzyme families was determined. Degradative enzyme families were previously described. (See, for example, da Luz et al. PLoS One 8: e69386 and DeIRe et al. Nature 592: 558-563.) This showed removal rates moderately correlated (correlation coefficients=0.3-0.4) with genes encoding protease, peroxidase, laccase, hydroxylase, and lipase functions (FIGS. 11A and 12A-12B), suggesting these enzymes were likely involved in the hybrid PSA degradation process.

The enzyme activities of protease, laccase, esterase, and lipase in the 23 fungal species were assayed using the liquid media assay. Overall, the activities of protease, laccase, and lipase, but not esterase, were significantly correlated with the adhesive removal rate (p<0.05; coefficients=0.43-0.47), which in general aligned with the genomic analysis. These positive correlations, to some degree, explained the PSA removal efficiency in fungi. For example, all three enzymes showed high levels in T. versicolor, T. pubesecens, and G. lucidum, which were among the best PSA degraders of those tested. In the other outstanding degraders such as P. microspora and P. chamaeropsis, high levels of protease and lipases were detected, which correlated with high PSA removal rates. However, there were exceptions that these three enzymes cannot explain. For example, these activities were relatively low for H. grisea and P. stipticus, even though these species demonstrated hybrid PSA removals of >50% (FIGS. 8A-8D). There were also species (e.g., T. hirsuta, G. trabeum, and Penicillium sp.) that can produce abundant protease, lipase, and/or laccase activities, but demonstrated low PSA-degrading efficiencies. These outliers suggest the involvement of other enzymes or degradative mechanisms in the degradation in addition to the above three enzymes. It also indicated that the degradative mechanisms of PSA may be variable among different species.

The analysis also showed that protease and palmitase activities were highly correlated (FIG. 11C; coefficients=0.74, p<0.05), indicating that these two enzymes tend to express together in the presence of PSA. These two enzymes may work synergistically in the degradation process. To test this, a regression analysis showed that the best model presented significant coefficients only for protease and laccase (FIG. 11D), which together explained 35% of the PSA degradation observed. No meaningful interactions between activities that contributed to the PSA degradation were found.

### Example 3. Fungal Consortia for Biodegradation of Hybrid Adhesive Polymers Containing High-Biomass Contents

This example shows that polymer-degrading fungal species and polymer-degrading fungal consortia were identified.

Methods

Use of consortia for the degradation of PSA. Lenzitus betulina ATF03, Trametes versicolor A1 ATF, Trametes pubescens Ban020, and Pestalotiopsis microspora were used to test fungal degradation. A liquid medium with the following composition was prepared: 1% Latex PSA containing 50% MM, 2.622 g/L NaH₂PO₄·H₂O, 1.219 g/L K₂HPO₄, 0.086 g/L (NH₄)₂SO₄, 0.735 g/L HOC(COONa)(CH₂COONa)₂·2H₂O (sodium citrate), 0.123 g/L MgSO₄·7H₂O, 0.500 g/L NH₄NO₃, 0.015 g/L CaCl₂·2H₂O, 0.05 mM FeCl₃, 0.014 mM ZnCl₂, 0.012 mM CoCl₂·6H₂O, 0.21 μM Na₂MoO₄·H₂O, 1 μM thiamine, 0.011 mM CuCl₂·2H₂O, 0.012 mM MnCl₂·4H₂O, and 0.012 mM H₃BO₃. The pH of the medium was adjusted to 5.6 and autoclaved at 121° C. for 20 minutes. 120-mL flasks containing 30 mL of media were inoculated with four 5-mm agar discs taken from the edge of actively growing mycelia on Malt Extract Agar (MEA). For the consortia experiments, the discs were distributed as indicated in the following parentheticals, L. betulina—T. pubescens (2 discs:2 discs), L. betulina—T. versicolor (2 discs:2 discs), and P. microspora—T. versicolor (3 discs:1 disc). Interaction assays between the strains were conducted previously to determine compatibility for co-culturing. The turbidity changes over time served as a proxy for PSA removal and was measured at an absorbance of 595 nm for 20 days. Each experiment was carried in triplicate.

PSA film fungal degradation under solid state culture (SSC) conditions. To test the ability of different fungal species and their consortia to degrade PSA films, seven biological treatments were used including single fungi and their consortia: 1) P. microspora, 2) T. versicolor, 3) T. pubescens, 4) L. betulina, 5) P. microspora—T. versicolor, 6) T. pubescens—L. betulina, and 7) T. versicolor—L. betulina. These fungi were cultured in mason jars using four different substrates: 1) 90 g soil mix (topsoil: peat moss: vermiculite, 1:1:1) +1 g of wood chips (birch); 2) 90 g soil mix (topsoil: peat moss: vermiculite, 1:1:1) +1 g wood chips (birch) +1% glucose; 3) 30 g wheat straw; and 4) 65 g wheat bran. All the substrates were saturated with water to field capacity (70% w/w). The samples were inoculated with twelve 10-mm agar discs taken from the edge of actively growing colonies on Potato Dextrose Agar (PDA). The number of discs used for the consortia is indicated in parenthesis, P. microspora (8)—T. versicolor (4), T. pubescens (6)—L. betulina (6), and T. versicolor (6)—L. betulina (6). The samples were incubated in the dark at 28° C. and 70% relative humidity for 100 days. The PSA films were buried 1 cm under the substrate surface. After the incubation time, the PSA films were recovered, and fungal mycelia and substrate residues were rinsed off the surface with distilled water. Subsequently, the films were dried at 70° C. overnight and their weight was recorded to calculate weight loss.

Results

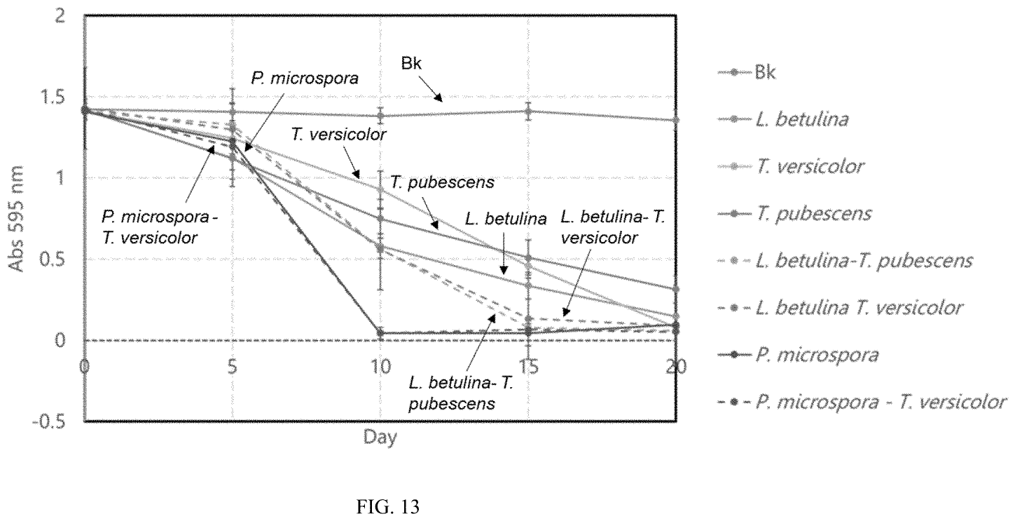

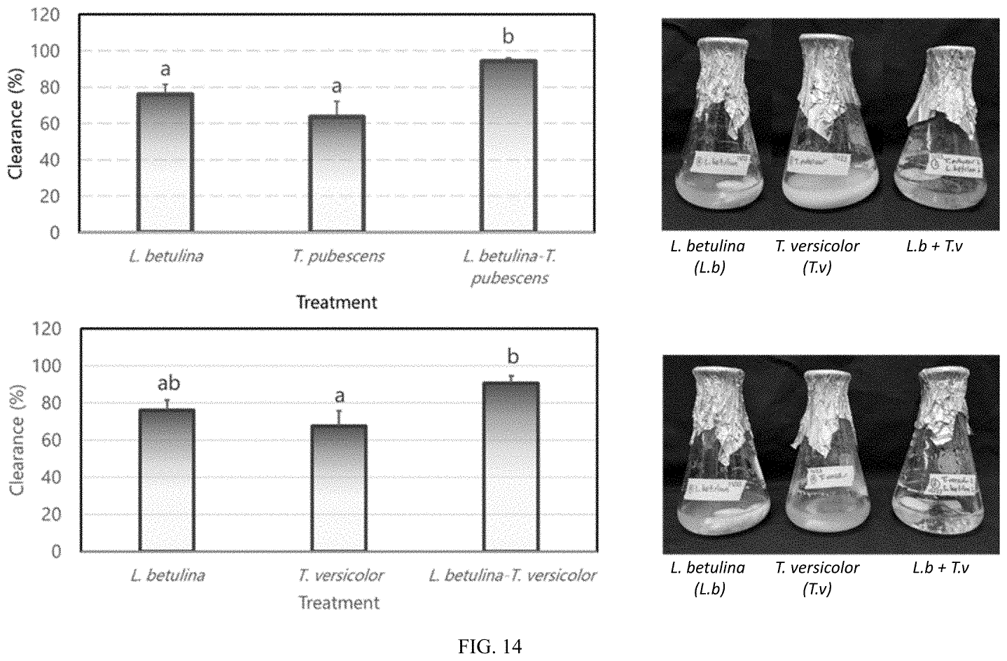

Use of consortia for the degradation of PSA. The results showed that the use of consortia accelerated the removal of PSA in liquid culture (FIG. 13). The effects are more evident at day 15 were the consortia for L. betulina—T. versicolor and L. betulina—T. pubescens outperformed the single-fungus cultures (FIG. 14). On the other hand, the consortia between P. microspora and T. versicolor did not show a better performance compared to the treatment with P. microspora alone. The consortia results, nonetheless, were not lower than those achieved by P. microspora which indicates that at the very least the presence of T. versicolor does not negatively affect PSA removal capacity.

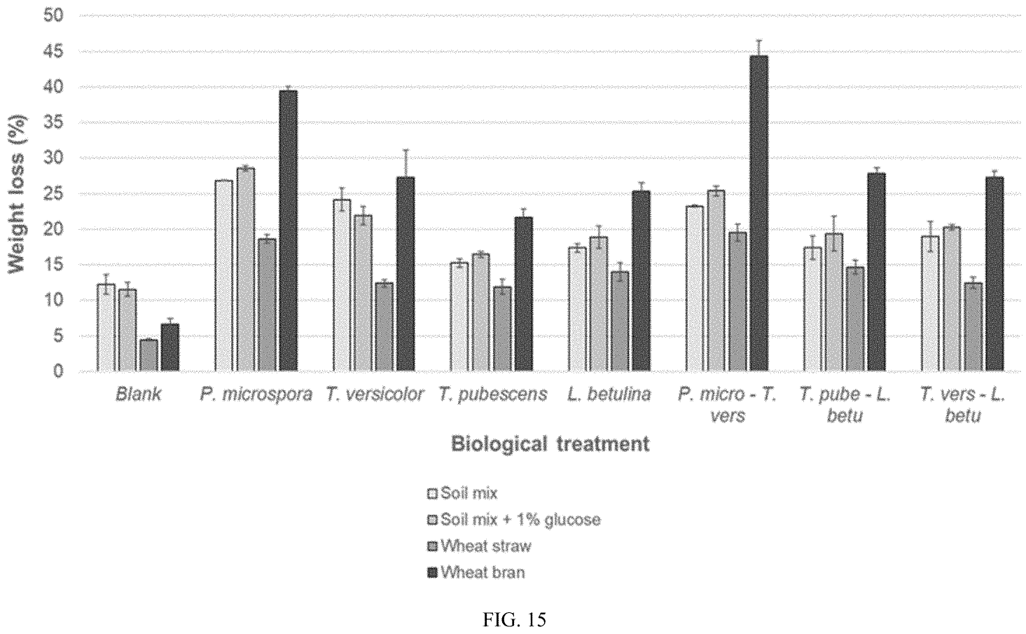

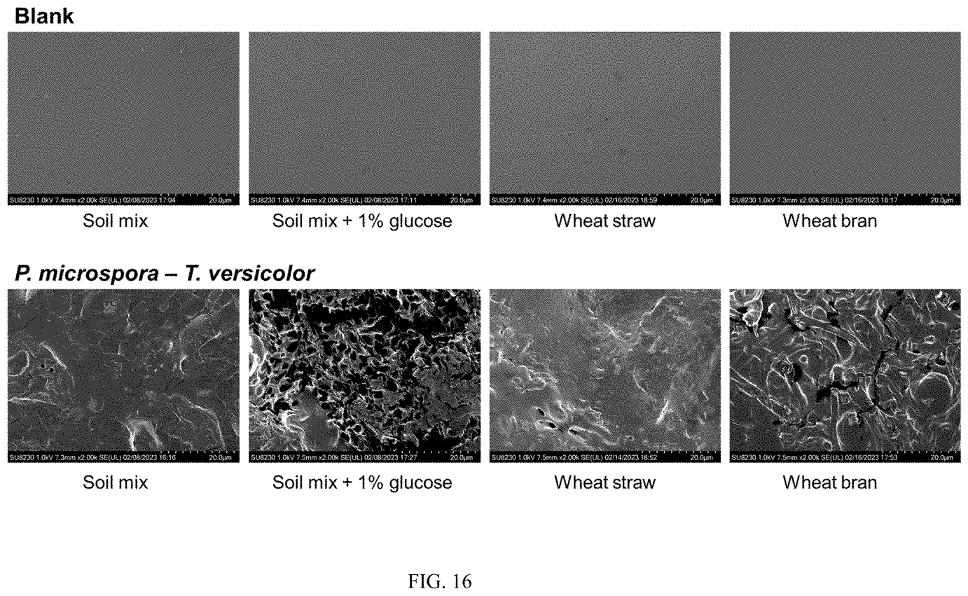

PSA film fungal degradation under solid state culture (SSC) conditions. The results in FIG. 15 showed that wheat bran was the best substrate of substrates tested with the dried PSA films being in contact with substrate for 100 days. Among the fungal species, the treatment using the consortium P. microspora—T. versicolor displayed the highest weight loss value (44.35%). FIG. 16 shows representative SEM images from some of the biological treatments tested in FIG. 15. The treatment using P. microspora—T. versicolor caused a damage and deterioration to the PSA film structure. For example, treatment using P. microspora—T. versicolor produced cracks, micropores, and holes. Thus this consortium constitutes a valuable option for the biodegradation of the PSA formulation using an inexpensive substrate such as wheat bran, which could facilitate disposal strategies for future commercial products developed with this adhesive formulation.

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of degrading a polymer, the method comprising:
contacting a polymer with one or more polymer-degrading fungal species,
wherein the polymer comprises a polyester graft, and
wherein the polymer is a polymerization product of at least a monomer selected from the group consisting of acrylates, methacrylates, and combinations thereof.

2. The method of claim 1, wherein the one or more polymer-degrading fungal species is selected from the group consisting of Pestalotiopsis chamaeropsis, Ganoderma lucidum, Gloeophylum trabeum, Lenzites betulina, Trametes pubescens, Trametes versicolor, Leucogyrophana olivascens, Plicaturopsis crispa, Fistulina hepatica, Armillaria sp., Lentinus edodes, Humicola grisea, Aurebasidium pullulans, Aspergillus niger, Penicillum sp., Coniophora puteana, Resinicium bicolor, Panellus stipticus, Trametes hirsuta, Ganoderma austrade, Fomitiporia mediterranea, and Pestalotiopsis micropsora.

3. The method of claim 2, wherein the one or more fungal species includes at least two fungal species selected from the group consisting of L. betulina and T. versicolor, L. betulina and T. pubescens, and P. microspora and T. versicolor.

4. The method of claim 1, wherein the polymer comprises at least 15 wt. % of the polyester graft.

5. The method of claim 1, wherein the polyester graft is a macromonomer.

6. The method of claim 5, wherein the macromonomer is a reaction product of at least a lactide monomer, a caprolactone monomer, or combinations thereof.

7. The method of claim 1, wherein the polymer is a pressure-sensitive adhesive.

8. The method of claim 1, wherein the polymer is a latex product.

9. The method of claim 1, comprising, following the contacting, degrading at least 40 wt. % of the polymer.

10. The method of claim 9, wherein the degrading occurs in a controlled environment, wherein the controlled environment does not contain other carbon-containing materials.

11. The method of claim 1, wherein the contacting further comprises adding wheat bran to the polymer and the polymer-degrading fungal species.

12. A method comprising:
contacting a polymer and one or more polymer-degrading fungal species, wherein the polymer comprises at least 15 wt. % of a polyester graft, wherein the polymer is a polymerization product of at least a monomer selected from the group consisting of acrylates, methacrylates, and
combinations thereof, and wherein the polymer comprises a copolymerizable lactide-based macromonomer; and
degrading the polymer with a predetermined time period.

13. The method of claim 12, wherein the predetermined time period is from about 1 day to about 30 days.

14. A method of degrading a polymer, the method comprising contacting:
a polymer with one or more polymer-degrading fungal species,
wherein the polymer comprises a polyester graft, and
wherein the polymer is a polymerization product of at least a monomer selected from the group consisting of acrylates, methacrylates, and combinations thereof.
a hydrolytic polypeptide selected from the group consisting of a protease and a lipase, and
an oxidative polypeptide selected from the group consisting of a laccase, a peroxidase, and a hydroxylase.

15. The method of claim 14, wherein the hydrolytic polypeptide, the oxidative polypeptide, or both is derived from a polymer-degrading fungal species.

16. The method of claim 15, wherein the one or more polymer-degrading fungal species is selected from the group consisting of Pestalotiopsis chamaeropsis, Ganoderma lucidum, Gloeophylum trabeum, Lenzites betulina, Trametes pubescens, Trametes versicolor, Leucogyrophana olivascens, Plicaturopsis crispa, Fistulina hepatica, Armillaria sp., Lentinus edodes, Humicola grisea, Aurebasidium pullulans, Aspergillus niger, Penicillum sp., Coniophora puteana, Resinicium bicolor, Panellus stipticus, Trametes hirsuta, Ganoderma austrade, Fomitiporia mediterranea, and Pestalotiopsis micropsora.

17. The method of claim 16, wherein the one or more fungal species includes at least two fungal species selected from the group consisting of L. betulina and T. versicolor, L. betulina and T. pubescens, and P. microspora and T. versicolor.

18. The method of claim 14, wherein the polyester graft is a macromonomer.

19. The method of claim 14, wherein the polymer is a pressure-sensitive adhesive.

20. The method of claim 14, wherein the polymer is a latex product.

21. The method of claim 14, wherein the degrading comprises a degradation of at least 40 wt. % of the polymer.
